(19) United States
(12) Patent Application Publication
(10) Pub. No.: US 2025/0082712 A1
(43) Pub. Date: Mar. 13, 2025

(54) WITHANIA SOMNIFERA AND ASPARAGUS RACEMOSUS IN TREATING POST-MENOPAUSAL CONDITIONS

(71) Applicant: NATREON, INC., New Brunswick, NJ (US)

(72) Inventor: Sanyasi R. KALIDINDI, Monroe, NJ (US)

(73) Assignee: NATREON, INC., New Brunswick, NJ (US)

(21) Appl. No.: 18/710,943

(22) PCT Filed: Nov. 15, 2022
(86) PCT No.: PCT/US2022/049905

§ 371 Date: May 16, 2024

Related U.S. Application Data
(60) Provisional application No. 63/280,356, filed on Nov. 17, 2021.

(51) Int. Cl. A61K 36/88; A61K 36/81; A61P 15/12

(52) U.S. Cl. CPC A61K 36/88; A61K 36/81; A61P 15/12

(57) ABSTRACT

Provided herein are methods and compositions for Asparagus racemosus and Withania somnifera in treating the quality of life, improving endothelial function and biomarkers, bone health biomarkers, and bone mineral density (BMD) in postmenopausal women.

### BACKGROUND

The invention generally relates to compositions and methods to treat post-menopausal conditions.

Menopause is a natural phenomenon which occurs as the age of a woman progresses. The unpleasant changes experienced by the subject during this phase is termed as menopausal syndrome which is characterized by psychosomatic disturbances. Hormone replacement therapy (HRT) is the conventional treatment. Hormonal preparations, especially estrogens, prescribed for relief of these symptoms are associated with adverse effects, sometimes serious. Phytoestrogens, also known as “dietary estrogens,” are a diverse group of non-steroidal plant-derived polyphenolic compounds. They exhibit structural similarity and mimic the effects of naturally occurring estrogen compounds in the body. A meta-analysis by Franco et al. [1] from 101 randomized clinical trials and 12 prospective non-randomized interventional or observational studies reported that use of Phytoestrogens reduced the signs and symptoms of menopause, especially hot flushes flashes, vaginal dryness, night sweats etc. They concluded that plant-based therapies effectively improve menopausal symptoms. They also suggested that rigorous studies are needed to determine their impact on menopausal health and symptom alleviation.

Review of literature revealed that medicinal herbs used in menopausal syndrome include Withania somnifera (Ashwagandha), Phyllanthus emblica (Amla), Asparagus racemosus (Shatavari), etc. In Ayurvedic system of medicine, Asparagus racemosus is frequently recommended to overcome stress-mediated reproductive health disorders. It has been prescribed routinely for menopausal symptoms, promote production of reproductive hormones, promote general wellbeing, etc. Pandey et al., [2] reported positive results in their study with Asparagus racemosus on improving female reproductive health, decreasing the symptoms of menopausal syndrome, and increasing antioxidant levels in the body.

Withania somnifera, (Ashwagandha), has a lot of medicinal properties. It is a well-known medicine for improving female reproductive health in many ways. Extract of Withania somnifera has been shown to exhibit its beneficial effects by decreasing oxidative stress. [3,4,5,6] It also is considered a rejuvenator. It helps in relieving pain associated with osteoporosis, nervous exhaustion, and muscular pains. Further, endothelial dysfunction is reported in menopausal women. Our earlier studies in diabetic and metabolic syndrome subjects have demonstrated that Withania somnifera produced significant improvement in the endothelial function and biomarkers. [7,8,9,10]

Compositions obtained from an extract of Withania somnifera plant have been described by the assignee of this disclosure, for example, U.S. Publication No. 2004/0166184, and U.S. Pat. Nos. 6,153,198 and 6,713,092.

The present invention attempts to solve these problems as well as others.

### SUMMARY OF THE INVENTION

Provided herein are methods and compositions for Asparagus racemosus and Withania somnifera in treating the quality of life, improving endothelial function and biomarkers, bone health biomarkers, and bone mineral density (BMD) in postmenopausal women.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

The below definitions and discussion are intended to guide understanding but are not intended to be limiting with regard to other disclosures in this application. References to percentage (%) in compositions of the present invention are to the % by weight of a given component to the total weight of the composition being discussed, also signified by “w/w”, unless stated otherwise.

“Administering”, “administration”, and the like, according to the present invention refer to providing a composition of the present invention to a subject so that the Asparagus racemosus, Withania somnifera, or a combination of Asparagus racemosus and Withania somnifera may reach the subject's bloodstream and/or tissues and/or cells, preferably skeletal muscle tissues and cells and associated tissues and cells, and act on the tissues and cells, bloodstream and overall body of the subject to increase the subject's muscular strength and/or muscular endurance. Administration may be by the subject or by another. Administration may be oral, for instance in the form of a dietary supplement, and/or in a solid dosage form, preferably in a discrete dose unit, such as a capsule including for instance the 125 mg Asparagus racemosus, Withania somnifera, or a combination of Asparagus racemosus and Withania somnifera capsule described below. Administration may also be through parenteral, intramuscular, transdermal, and other physiologically acceptable routes. In the below Example or as described elsewhere herein, supplementation with Asparagus racemosus, Withania somnifera, or a combination of Asparagus racemosus and Withania somnifera is administration according to the present invention. A “subject” according to this invention is a human (female, adult) or other mammals such as a monkeys, apes, gorillas, primates, shrews, or other mammals where treatment of PMS may be desired.

In the present disclosure, an “effective amount” of Asparagus racemosus, Withania somnifera, combination of Asparagus racemosus and Withania somnifera, or composition refers to an amount of Asparagus racemosus or Withania somnifera that, once administered to a subject, will reach the subject's bloodstream and/or bodily tissues.

A “composition” of the present invention comprises Asparagus racemosus, Withania somnifera, or a combination of Asparagus racemosus and Withania somnifera. A composition of the present invention may comprise, consist essentially of, or consist of, Asparagus racemosus, Withania somnifera, or a combination of Asparagus racemosus and Withania somnifera. In one embodiment, Asparagus racemosus may be an extract of Asparagus racemosus such as an aqueous extract. In the present invention, an “aqueous extract” is prepared by disrupting Asparagus racemosus from its natural state and treating the disrupted Asparagus racemosus with water or aqueous solution to form the aqueous extract. A “standardized aqueous extract” is an extract in which specific components have been identified and are present in a minimum or maximum amount or a specific range, so as to render the extract consistent at least with regard to those components from one batch to the next. In an embodiment, a composition according to the present invention comprises a standardized aqueous extract of Asparagus racemosus. In an embodiment, an Asparagus racemosus extract of this invention includes at least 40% saponins. A composition of the present invention may be a blend of Asparagus racemosus (e.g. standardized powdered aqueous extract of Asparagus racemosus) for instance with microcrystalline cellulose, croscarmellose sodium, and silicon dioxide as discussed below and as provided in the 125 mg capsules described in the below Example. A composition of the present invention may be formulated into nutraceutical or pharmaceutical dosage forms comprising for instance tablets, capsules, powders, liquids, chews, gummies, transdermals, injectables, dietary supplements, topical creams, lozenges, pills, and so forth. A composition of the present invention may further comprise one or more excipients, additives, and/or other substances, including for instance microcrystalline cellulose, croscarmellose sodium, magnesium stearate, and/or silicon dioxide. In the below Example or as described elsewhere herein, a supplement such as a 125 mg or a 250 mg Asparagus racemosus capsule, a 125 mg or a 250 mg Withania somnifera capsule, or a combination of a 125 mg Asparagus racemosus and a 125 mg Withania somnifera capsule is a composition of the present invention.

A “dietary supplement” according to the present invention refers to a composition comprising Asparagus racemosus, Withania somnifera, or a combination of Asparagus racemosus and Withania somnifera according to the present invention which is administered as an addition to a subject's diet over a period of time. In an embodiment, a dietary supplement containing an effective amount of Asparagus racemosus, Withania somnifera, or a combination of Asparagus racemosus and Withania somnifera according to the present invention is administered orally. In an embodiment, the dietary supplement is administered daily; in an embodiment, the dietary supplement is administered daily for 1-4, 1-8, 1-12, 4-8, 8-12, 1-24, 4-24, 8-24, or 12-24 weeks, or more, or for another period of time according to the present invention. A dietary supplement may be formulated into various forms, such as powder, liquid, pill, capsule, or tablet, as discussed throughout this application.

The compounds of the present invention may take the form of salts. The term “salts” embraces addition salts of free acids or free bases which are compounds of the invention. The term “pharmaceutically-acceptable salt” refers to salts which possess toxicity profiles within a range that affords utility in pharmaceutical applications.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoroacetic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. In the present examples of compounds of Asparagus racemosus, Withania somnifera, or a combination of Asparagus racemosus and Withania somnifera, i.e., compounds containing amino groups, said compounds can be isolated as salts of inorganic acids or strong organic acids, e.g. hydrochloric acid or trifluoroacetic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine), tromethamine (tris(hydroxymethyl)aminomethane), and procaine.

All of these salts may be prepared by conventional means from the corresponding compound of Asparagus racemosus, Withania somnifera, or a combination of Asparagus racemosus and Withania somnifera by reacting, for example, the appropriate acid or base with the compound Asparagus racemosus, Withania somnifera, or a combination of Asparagus racemosus and Withania somnifera. Preferably the salts are in crystalline form, and preferably prepared by crystallization of the salt from a suitable solvent. The person skilled in the art will know how to prepare and select suitable salts forms for example, as described in Handbook of Pharmaceutical Salts: Properties, Selection, and Use by P. H. Stahl and C. G. Wermuth (Wiley-VCH 2002).

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. In accordance with one embodiment, suitable nutraceutically acceptable carriers can include ethanol, aqueous ethanol mixtures, water, fruit and/or vegetable juices, and combinations thereof.

The term “preparation” is intended to include the formulation of the active compound with encapsulating material as carrier providing a capsule in which the active component, with or without carriers, is surrounded by a carrier, which is thus in association with it. Tablets, powders, capsules, pills, sachets, and lozenges are included. Tablets, powders, capsules, pills, sachets, and lozenges can be used as solid forms suitable for oral administration.

### Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films or oral dissolvable strips can be used. Other useful delivery systems comprise oral or nasal sprays or inhalers, and the like.

For oral administration, Asparagus racemosus, Withania somnifera, or a combination of Asparagus racemosus and Withania somnifera extract may be further combined with one or more solid inactive ingredients for the preparation of tablets, capsules, pills, powders, granules or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

The components of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof many comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably contain from five or ten to about seventy percent of the active compound(s). Suitable carriers are microcrystalline cellulose, sugar, lactose, pectin, dextrin, starch, gelatin, tragacanth, methylcellulose, sodium carboxymethlycellulose, a low melting wax, cocoa butter, and the like, and other excipients may include magnesium stearate, stearic acid, talc, silicon dioxide, etc.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Compositions suitable for topical administration in the mouth includes lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compound will generally have a small particle size for example of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenges itself, or it can be the appropriate number of any of these in packaged form.

Tablets, capsules and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of Remington's Pharmaceutical Sciences (Mack Publishing Co., Easton, Pa.).

Solid nutritional compositions for oral administration may optionally contain, in addition to the above enumerated nutritional composition ingredients or compounds: carrier materials such as corn starch, gelatin, acacia, microcrystalline cellulose, kaolin, dicalcium phosphate, calcium carbonate, sodium chloride, alginic acid, and the like; disintegrators including, microcrystalline cellulose, alginic acid, and the like; binders including acacia, methylcellulose, sodium carboxymethylcellulose, polyvinylpyrrolidone, hydroxypropyl methylcellulose, ethyl cellulose, and the like; and lubricants such as magnesium stearate, stearic acid, silicone fluid, talc, waxes, oils, colloidal silica, and the like. The usefulness of such excipients is well known in the art.

Liquid nutritional compositions for oral administration in connection with a method for preventing and/or treating inflammation, colds and/or flu can be prepared in water or other aqueous vehicles. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir including or containing, together with the above enumerated ingredients or compounds, wetting agents, sweeteners, and coloring and flavoring agents. Various liquid and powder nutritional compositions can be prepared by conventional methods. Various ready-to-drink formulations (RTD's) are contemplated.

### Routes of Administration

The compositions may be administered by any suitable route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebal, intraocular injection or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in form of shaped artices, e.g. films or microcapsules.

The methods described above may be further understood in connection with the following Examples. In addition, the following non-limiting examples are provided to illustrate the invention. The results of an extraction process depend upon the solvent used, temperature of extraction and duration of the extraction process. In several embodiment of this invention, these factors can be optimized to isolate and/or enrich and preserve the bioactives of Asparagus racemosus, Withania somnifera, or a combination of Asparagus racemosus and Withania somnifera.

### DESCRIPTION

The present methods are directed to administration of Asparagus racemosus, Withania somnifera, or a combination of Asparagus racemosus and Withania somnifera to significantly increase biomarkers of bone health. The biomarkers of bone health may be selected from C-terminal telopeptide-1 (CTX-1), Bone Alkaline Phosphatase (BAP), Osteoprotegerin (OPG) and serum receptor activator of nuclear factor-κB ligand (sRANKL). The administration of Asparagus racemosus in combination with Withania somnifera significantly increases biomarkers of stress and endothelial function. The biomarkers of stress may be selected from Malondialdehyde (MDA), Nitric Oxide (NO), and Glutathione (GSH). Endothelial function may be estimated by improvement in reflection index (RI.) Significant improvement in quality of life as measured by MENQOL questionnaire was also observed with Asparagus racemosus and Withania somnifera administration.

Also, the quality of life as measured by MENQOL score significantly increases with the administration of Asparagus racemosus and Withania somnifera over a period of at least 4 weeks, for instance as shown in the Example below. Further, the and administration of Asparagus racemosus and Withania somnifera administration increases endothelial function as estimated by improvement in reflection index (RI) treating for at least 8 weeks and statistically significant increased endothelial function as estimated by improvement in reflection index (RI) with administration of Asparagus racemosus and Withania somnifera for at least 12 and 24 weeks, for instance as shown in the below Example. The biomarkers of stress—NO, GSH, and MDA significantly improved with the administration of Asparagus racemosus and Withania somnifera when compared to baseline at 12 and 24-weeks. In one embodiment, administration of Asparagus racemosus and Withania somnifera improves NO and GSH levels in at least 12 and at least 24-weeks. In another embodiment, administration of Asparagus racemosus and Withania somnifera improved the biomarker MDA at least 24-weeks of treatment. In one embodiment, administration of Withania somnifera improves the stress biomarkers NO, GSH, and MDA at least 12 weeks of treatment and at least 24-weeks of treatment. In one embodiment, the administration of Asparagus racemosus and Withania somnifera does not increase platelet aggregation and does not increase serious adverse events over at least 12 weeks and at least 24 weeks of treatment.

In an embodiment, a standardized, powdered, aqueous extract of Asparagus racemosus includes one or more of the following characteristics: Appearance: a free flowing powder, yellowish-brown in color, with a slightly bitter taste, and a water-soluble extractive value of about 85-95%, for instance 90-93%, for instance 91.38% (w/w). By assay, total saponins (by gravimetry) of the standardized powdered aqueous extract of Asparagus racemosus are at least 40% (w/w) of the extract, for instance about 40-60%, for instance 45-50%, for instance 48-49% (w/w), for instance 48.4% (w/w); total amino acids (including Arginine) of about 4-8%, for instance 5-6% (w/w), for instance 5.42%; moisture content (by Karl-Fischer titration) of about 1-8%, for instance 5-6%, for instance 5.80% (w/w); sulfated ash content of about 1-8%, for instance 3-4% (w/w), for instance 3.96%; particle size of the powdered extract passing through mesh #40 of 75-100%, for instance 100% and particle size passing through mesh #80 of about 80-100%, for instance 97-99%, for instance 98.30%; bulk density of about 0.2-0.5 grams/cubic cm, for instance 0.3-0.4 grams/cubic cm, for instance 0.384 gm/cc; and tapped density of about 0.3-0.6 grams/cubic cm, for instance 0.4-0.5 grams/cubic cm, for instance 0.425 gm/cc. In an embodiment, the standardized, powdered, aqueous extract of Asparagus racemosus is the Asparagus racemosus extract included in the 125 mg or 250 mg capsules used in the below Example. In an embodiment, Asparagus racemosus extract is stored in sealed containers at 15° C. to 25° C. In an embodiment, said storage avoids light. In an embodiment, the powdered Asparagus racemosus extract described above is stable for at least 3 years.

In an embodiment, a capsule of the present invention includes Asparagus racemosus, for instance a standardized, powdered, aqueous extract of Asparagus racemosus such as identified above. In an embodiment, a capsule such as the 125 mg or the 250 mg Asparagus racemosus capsule identified in the below Example is prepared as follows: standardized powdered aqueous extract of Asparagus racemosus is blended in amounts such as described in the below Example with microcrystalline cellulose, croscarmellose sodium, and silicon dioxide in a blender such as a V-blender, equipped with SIFT-N-BLEND, for instance for 5 minutes without using SIFT-N-BLEND and then 10 minutes using SIFT-N-BLEND at 1500 RPM. Next, magnesium stearate is added and blended for instance without SIFT-N-BLEND for 5 minutes. Then, the Asparagus racemosus blend is discharged from the blender into for instance a tared double poly bag lined HDPE drum, the bags tied shut, and the blend weighed and sealed into the drum. In an embodiment, the encapsulation of an Asparagus racemosus blend is in a room with humidity of not more than 40% RH (relative humidity). In an embodiment, a capsule contains about 316 mg of the Asparagus racemosus blend and weighs in total about 390 mg±3%. In an embodiment, the Asparagus racemosus blend is stored in original, sealed containers at 15° C. to 25° C. In an embodiment, said storage avoids light. In an embodiment, the powdered Asparagus racemosus blend described above is stable for at least 3 years. In an embodiment, a capsule containing 250 mg Asparagus racemosus in a blend as described above is white and opaque and in an embodiment comprises at least 30% (w/w) saponins.

A placebo capsule of the present invention may comprise one or more of microcrystalline cellulose, croscarmellose sodium, silicon dioxide, and magnesium stearate. For instance, a placebo capsule may include these ingredients in amounts defined in the Example below. A placebo capsule of the present invention may be prepared as follows: blend microcrystalline cellulose, croscarmellose sodium, and silicon dioxide in a blender such as a V-blender, equipped with SIFT-N-BLEND, for instance for 5 minutes without using SIFT-N-BLEND and then 10 minutes using SIFT-N-BLEND at 1500 RPM. Next, magnesium stearate is added and blended for instance without SIFT-N-BLEND for 5 minutes. Then, the blend is discharged from the blender into for instance a tared double poly bag lined HDPE drum, the bags tied shut, and the placebo blend weighed and sealed into the drum. In an embodiment, the encapsulation of a placebo blend is in a room with humidity of not more than 40% RH (relative humidity). In an embodiment, a capsule contains about 316 mg of the placebo blend and weighs in total about 390 mg±3%. In an embodiment, the placebo blend is stored in sealed containers at 15° C. to 25° C. In an embodiment, said storage avoids light.

In an embodiment, an effective amount of Asparagus racemosus is at least 125 mg/day, 250 mg/day, or 500 mg/day, for instance in an adult human subject, when administered in combination with an effective amount of discussed in the Example below. In an embodiment, an effective amount of Asparagus racemosus according to this invention is 50-1000 mg/day, for instance 50, 100, 200, 300, 400, 500, 600, 700, 800, 900, or 1000 mg/day, or any amount or range within said range. In an embodiment, an effective amount of Asparagus racemosus of this invention may be about 0.5 to about 20 mg/kg body weight, or another amount in view of the body weight of the subject. In an embodiment, an effective amount of Asparagus racemosus is administered to a subject daily. In an embodiment, the Asparagus racemosus may be administered for instance every day, twice a day, or three times a day, according to this invention.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Evaluation of the Effect of Extracts of Withania somnifera and Asparagus racemosus on Quality of Life, Endothelial Function, and Biomarkers in Post-Menopausal Women

### Objectives:

Primary objective: 1) to compare the efficacy of 24-week treatment with 125 mg and 250 mg extract of Withania somnifera (Ashwagandha), 125 and 250 mg extract of Asparagus racemosus, and a combination of Withania somnifera (Sensoril) 125 mg+Asparagus racemosus 125 mg, and placebo given twice daily, on quality of life using MENQOL questionnaire; 2) to compare the efficacy of 24-week treatment with the six treatment groups given twice daily on endothelial function as measured by estimation of reflection index (RI), bone mineral density (BMD) and bone health biomarkers (CTX, BAP, OPG, sRANKL),

Secondary objective: 1) to compare the efficacy of 24-week treatment with the six treatment groups given twice daily on biomarkers of oxidative stress (MDA, GSH, NO); inflammatory biomarker (hsCRP), and platelet aggregation; 2) to compare the efficacy of 24-week treatment with the six treatment groups given twice daily on lipid profile; and 3) to evaluate the safety and tolerability of the above products.

STUDY DESIGN: Randomized, double blind, placebo controlled, parallel-group study.

STUDY POPULATION: Postmenopausal women primarily from the Gynecology outpatient department of NIMS were recruited into the study. Study was conducted in the Dept. of CP&T, NIMS.

Inclusion criteria: 1) Women of age between 40-55 years; 2) Amenorrhea for ≥12 months—where menopause is defined as having no menstrual period for 12 consecutive months by The American College of Obstetricians and Gynecologists—ACOG; 3) Normal function of thyroid, liver, and kidney; 4) Serum 25-hydroxy vitamin D≥20 ng/mL; 5) No bisphosphonates at all if they had a treatment lasting at least 12 months; and 6) Willing to participate in the study for 24 weeks.

Exclusion criteria: 1) Subjects with evidence/history of malignancy; 2) Surgical menopause subjects; 3) Established cases of mental illness, hypertension, diabetes mellitus, rheumatoid arthritis, coronary artery disease, hepatic, and renal impairment; 4) History of, or evidence for, metabolic bone disease including recent fractures; 5) Having received medication (calcitonin, raloxifene or systemic glucocorticoids) within 3 months of the study initiation; 6) Having hormone/hormone-like replacement therapy within 6 months of the study initiation; 7) Glycated hemoglobin of ≥8% in the past 3 months; 8) History of use of statins or other drugs for cholesterol control within 3 months of the study initiation; 9) Chronic smoking and/or regular alcohol intake; and 10) Body mass index (BMI) less than 20 or greater than 32.

STUDY MEDICATION: After screening, all the eligible subjects were randomized to either of the six treatment groups in a double-blind manner: Group A—Extract of Withania somnifera (Sensoril) 125 mg—one capsule twice daily; Group B— Extract of Withania somnifera (Sensoril) 250 mg—one capsule twice daily; Group C—Extract of Asparagus racemosus 125 mg—one capsule twice daily; Group D—Extract of Asparagus racemosus 250 mg—one capsule twice daily; Group E—Withania somnifera (Sensoril) 125 mg+A. racemosus 125 mg—one capsule twice daily; and Group F—identical placebo—one capsule twice daily. All the subjects were instructed to take one capsule daily in the morning and night after food for a period of 24 weeks.

### Study Procedure:

The study was conducted following the Declaration of Helsinki (2013) and ‘Guidelines for Clinical Trials on Pharmaceutical Products in India—GCP Guidelines issued by the Central Drugs Standard Control Organization, Ministry of Health, and Government of India, and New Drugs and Clinical Trials Rule, 2019, India. Institutional ethics committee approval was obtained before starting the study. The study has been registered in the clinical trial registry, India.

Subjects were referred from the Gynecology outpatient department of NIMS for screening and enrollment with the present study. After written informed consent and screening, eligible subjects were randomly assigned to one of the six treatment groups in a double-blind fashion. All the subjects were instructed to take one capsule of the allocated medicine daily in the morning and night after food for a period of 24 weeks. The study drugs were dispensed by the pharmacist. Subjects were requested to come for follow-up visits at 4, 8, 12- and 24-weeks post randomization.

At screening visit or visit 1, after describing in detail the study to the participants, a signed informed consent was obtained from them. Then subject's demography and medical history were taken, and their vitals recorded. Endothelial function was assessed. Blood samples were drawn for safety lab parameters and vitamin D.

Subjects were asked to report to the unit within one week for visit 2, which was the randomization visit. Subjects were assessed for inclusion/exclusion criteria followed by physical examination and then randomized to one of the treatment arms. The study medication was dispensed as per the randomization schedule and the subjects requested to report for next follow up visit after four weeks (visit 3). Review of adverse events was done during the visit. All the procedures included in the schedule of visits was followed—MENQOL questionnaire, biomarkers of bone health and oxidative stress, hsCRP, and platelet aggregation test using ADP and collagen. DEXA scan for estimation of bone mineral density (BMD) was done at this visit.

In visit 3 (Week 4), subjects were reviewed, general examination results and vital signs recorded. Any concomitant medication intake and any adverse reactions reported was recorded in the case record form. Subject's medication compliance was monitored by pill count method. MENQOL questionnaire was administered and endothelial function evaluated.

At visit 4 (week 8), general examination was done, and vital signs recorded. Any concomitant medication intake and any adverse reactions reported was recorded in the case record form. Subject's medication compliance was monitored by pill count method. MENQOL questionnaire was administered and endothelial function evaluated. Subjects was then asked to report for visit 5 (week 12).

At visit 5 (week 12), general examination was done, and vital signs recorded. Any concomitant medication intake and any adverse reactions reported was recorded in the case record form. Subject's medication compliance was also assessed by pill count method. MENQOL questionnaire was administered and endothelial function evaluated. Blood samples was collected and archived for future biomarker assessments as per the schedule of visits table. Subjects was then be asked to report for visit 6 (week 24) i.e., end of treatment visit.

At visit 6 (week 24—end of treatment) all vital parameters were recorded; general and physical examination was performed. Subject's medication compliance was evaluated. They were enquired regarding any adverse drug reaction and recorded in case record form. MENQOL questionnaire was administered and endothelial function evaluated. Blood samples were collected for estimation of biomarkers of bone health, oxidative stress, hsCRP, and platelet aggregation test using ADP and collagen, and safety assessments as shown in schedule of visits table. DEXA scan for BMD was done.

VISIT SCHEDULE

| | VISIT | | | | | |
|---|---|---|---|---|---|---|
| | | | | | | Visit 6 |
| | Visit 1 | Visit 2 | Visit 3 | Visit 4 | Visit 5 | End of |
| | Screening | Randomization | Follow up | Follow up | Follow up | treatment |
| | Week | | | | | |
| Procedure | −1 | 0 | 4 | 8 | 12 | 24 |
| Informed Consent | X | | | | | |
| Assessment of Inclusion & | X | X | | | | |
| Exclusion criteria | | | | | | |
| Demography | X | | | | | |
| Medical & surgical history | X | | | | | |
| Physical examination | X | X | X | X | X | X |
| Height | X | | | | | |
| Weight | X | X | X | X | X | X |
| Vital signs | X | X | X | X | X | X |
| QOL Assessment | | X | X | X | X | X |
| MENQOL Questionnaire | | | | | | |
| Evaluation of endothelial | X | | X | X | X | X |
| function: RI | | | | | | |
| Evaluation of Bone | | X | | | X | X |
| Biomarkers: | | | | | | |
| CTX, BAP, OPG, sRANKL | | | | | | |
| BMD (Bone mineral | | X | | | | X |
| density) DEXA scan | | | | | | |
| Evaluation of Oxidative | | X | | | X | X |
| stress biomarkers: MDA, | | | | | | |
| GSH, NO | | | | | | |
| Evaluation of systemic | | X | | | X | X |
| inflammation: hsCRP | | | | | | |
| Lipid Profile | X | | | | | X |
| Platelet aggregation Test | X | | | | | X |
| (ADP & Collagen) | | | | | | |
| Vitamin D | X | | | | | |
| Biochemical Parameters: | X | | | | | X |
| Safety assessment, | | | | | | |
| Dispense of Drug | | X | X | X | X | |
| Drug Accountability | | | X | X | X | X |
| Concomitant Medication | X | X | X | X | X | X |
| AE recording | | X | X | X | X | X |
| SAE recording | | X | X | X | X | X |

### Methods of Assessment:

Quality of life: It was measured using MENQOL—The Menopause-specific Quality of Life Questionnaire. Appropriate permissions have been obtained for the use of questionnaire.

Assessment of Endothelial function: Salbutamol Challenge Test

A salbutamol challenge test employing digital volume plethysmography was used to assess endothelial function as reported by Chowienezyk et al. [11] and Naidu et al. [12] Patients were examined in supine position after 5 minutes of rest. A digital volume pulse (DVP) was obtained using photo plethysmograph (Pulse Trace PCA2, PT200, Micro Medical, Kent, UK) transmitting infrared light at 940 nm, placed on the index finger of right hand. The signal from the plethysmograph was digitized using a 12-bit analogue to digital converter with a sampling frequency of 100 Hz. DVP waveforms was recorded over 20 second period and the height of the late systolic/early diastolic portion of the DVP was expressed as a percentage of the amplitude of the DVP to yield the reflection index (RI), as per the procedure described in detail by Millaesseau et al. [13] DVP recordings was taken, three measurements of reflection index (RI) were calculated, and the mean value was determined. Patients were administered 400 mcg of salbutamol by inhalation. After 15 minutes three measurements of RI were obtained again and the difference in mean RI before and after administration of salbutamol was used for assessing endothelial function. A change of ≤6% in RI post salbutamol was considered as endothelial dysfunction.

### Evaluation of Biomarkers, Platelet Aggregation Test and Safety Parameters

The biomarkers of bone health C-terminal telopeptide (CTX, a bone resorption marker), serum bone-specific alkaline phosphatase (BAP, a bone formation marker), serum receptor activator of nuclear factor-KB ligand (sRANKL), and serum osteoprotegerin (OPG), were estimated using commercially available kits by Elisa method. The levels of Nitric oxide [14], MDA [15], Glutathione [16] were estimated spectrophotometrically and hsCRP (high sensitivity C-reactive protein) by ELISA method. Samples were collected after an overnight fast for determination of hemoglobin, blood urea and serum creatinine, liver function test, lipid profile [Total cholesterol, High density lipoprotein cholesterol (HDL-C), low density lipoprotein cholesterol (LDL-C), very low-density lipoprotein (VLDL-C) and Triglycerides] using appropriate standard techniques. Platelet aggregation test with ADP and Collagen was done using platelet aggregometry test i.e., (Chronolog Light Transmittance Aggregometry).

### Outcome Measures:

Primary: 1) Changes in QOL using MENQOL questionnaire with 24 weeks treatment compared to baseline; 2) Changes in RI at the end of 24 weeks treatment compared to baseline; and 3) Changes in biomarkers of bone health (CTX, BAP, OPG, sRANKL) and BMD at the end of 24 weeks treatment compared to baseline.

Secondary: 1) Changes in biomarkers of oxidative stress (MDA, GSH, NO); inflammatory biomarker-hsCRP, and platelet aggregation at 24 weeks treatment compared to baseline; 2) Changes in lipid profile at 24 weeks treatment compared to baseline; 3) Safety and tolerability assessed at the end of 24 weeks treatment compared to baseline.

### Statistical Analysis:

To detect a clinically significant difference between the group means of MENQOL questionnaire at the end of 24 weeks treatment for a 95% confidence interval with standard deviation of 7 and acceptable margin of error 3, with 10% of screen failure and 10% dropout rate a total of 144 subjects would be screened to get 120 subjects who complete the study, with 20 subjects in each of the groups.

Study data was expressed as mean±SD. Data was analysed for normal distribution. One-Way ANOVA test was used for between the group analyses for the data set which followed normal distribution. The data set which did not follow normal distribution was analysed using nonparametric tests: Kruskal-Wallis and Dunn's multiple comparisons test for between the group analyses. Within group analysis was done using repeated measures one-way ANOVA for data set following normal distribution. Friedman test and Dunn's multiple comparisons test was performed for the data set not following normal distribution for within group analysis. A p-value≤0.05 was considered statistically significant.

### Results:

A total of 135 subjects were screened, and 127 eligible subjects enrolled in the study. A total of 123 subjects completed 24 weeks of treatment. Two subjects from group C (Asparagus 125 mg BD), one subject from group D (Sensoril 125 mg BD+Asparagus 125 mg BD), and one subject from group F (Asparagus 250 mg BD) dropped out of the study before the first follow-up citing logistical reasons. The demographic data in Table 1 shows all the subjects who completed the study. Table 2 to Table 12 includes the data of the subjects who have completed the study. Thus, a total of 20 subjects in group A (Placebo BD), 20 subjects in group B (Sensoril 125 mg BD), 20 subjects in group C (Asparagus 125 mg BD), 21 subjects in group D (Sensoril 125 mg BD+Asparagus 125 mg BD), 22 subjects in group E (Sensoril 250 mg BD) and 20 subjects in group F (Asparagus 250 mg BD) completed 24 weeks of study treatment.

TABLE 1
Demographic Data of all the randomized subjects:

| | | | | Sensoril | | |
|---|---|---|---|---|---|---|
| | | | | 125 mg BD + | | |
| | | Sensoril | Asparagus | Asparagus | Sensoril | Asparagus |
| | Placebo BD | 125 mg BD | 125 mg BD | 125 mg BD | 250 mg BD | 250 mg BD |
| | (A) | (B) | (C) | (D) | (E) | (F) |
| Total No. | 20 | 20 | 20 | 21 | 22 | 20 |
| Age | 50.90 ± 3.13 | 50.10 ± 2.95 | 51.10 ± 3.29 | 51.00 ± 3.15 | 50.55 ± 2.46 | 51.30 ± 2.99 |
| (Years) | | | | | | |
| BMI | 26.20 ± 2.20 | 26.65 ± 3.13 | 25.78 ± 2.45 | 26.61 ± 2.62 | 27.02 ± 1.82 | 26.60 ± 1.87 |
| (Kg/m2) | | | | | | |

The demographic characteristics of all the six study groups are depicted in Table 1. There were no significant differences between treatment groups in baseline characteristics, including age and body mass index (BMI) indicating a homogenous population.

TABLE 2
MENQOL SCORE (The Menopause-Specific Quality of Life score) from
baseline to 4, 8, 12 and 24-weeks of treatment (Mean ± SD)

| Treatment | | 4- | % | 8- | % | 12- | % | 24- | % |
|---|---|---|---|---|---|---|---|---|---|
| Groups | Baseline | weeks | change | weeks | Change | weeks | change | weeks | Change |
| Placebo BD | 5.15 ± | 5.15 ± | −0.09 ± | 5.14 ± | −0.19 ± | 5.14 ± | −0.36 ± | 5.11 ± | −0.85 ± |
| (A) | 0.51 | 0.50 ns | 0.51 | 0.51 ns | 0.34 | 0.51 ns | 0.78 | 0.50 ns | 1.73 |
| | | | | NS | | NS | | NS | |
| Sensoril | 5.14 ± | 5.02 ± | −2.42 ± | 4.82 ± | −6.23 ± | 4.60 ± | −10.43 ± | 4.24 ± | −17.68 ± |
| 125 mg BD | 0.60 | 0.63 | 2.39 | 0.64 * | 3.98 | 0.61 * | 4.35 | 0.62 * | 4.05 |
| (B) | | $ NS | | NS | | NS | | β | |
| Asparagus | 4.94 ± | 4.88 ± | −1.12 ± | 4.71 ± | −4.49 ± | 4.58 ± | −7.03 ± | 4.28 ± | −13.22 ± |
| 125 mg BD | 0.50 | 0.49 ns | 1.85 | 0.47 @ | 4.80 | 0.43 * | 6.86 | 0.45 * | 7.14 |
| (C) | | NS | | NS | | NS | | Ω | |
| Sensoril | 5.20 ± | 5.09 ± | −2.01 ± | 4.92 ± | −5.42 ± | 4.73 ± | −8.94 ± | 4.44 ± | −14.66 ± |
| 125 mg + | 0.51 | 0.52 * | 1.68 | 0.52 * | 2.94 | 0.53 * | 4.56 | 0.56 * | 4.15 |
| Asparagus | | NS | | NS | | NS | | ‡ | |
| 125 mg BD | | | | | | | | | |
| (D) | | | | | | | | | |
| Sensoril | 5.40 ± | 5.13 ± | −4.56 ± | 4.92 ± | −8.58 ± | 4.49 ± | −16.43 ± | 4.13 ± | −23.02 ± |
| 250 mg BD | 0.71 | 0.62 * | 4.79 | 0.58 * | 5.59 | 0.55 * | 7.13 | 0.47 * | 6.22 |
| (E) | | NS | | NS | | Ω | | ∞ | |
| Asparagus | 5.10 ± | 4.89 ± | −4.25 ± | 4.66 ± | −8.61 ± | 4.42 ± | −13.36 ± | 4.04 ± | −20.83 ± |
| 250 mg BD | 0.49 | 0.51 * | 2.50 | 0.51 * | 5.14 | 0.58 * | 5.87 | 0.46 * | 4.83 |
| (F) | | NS | | NS | | Ω | | ∞ | |

| | |
|---|---|
| At 4-weeks when compared to | BETWEEN GROUP ANALYSIS |
| baseline: | At 4-weeks: |
| Group A-ns | A vs B, A vs C, A vs D, A vs E, A vs F, B vs C, B vs D, B |
| Group B-$-p ≤ 0.01 | vs E, B vs F, C vs D, C vs E, C vs F, D vs E, D vs F, E vs F- |
| Group C-ns | NS |
| Group D, Group E, Group F-*-p ≤ 0.0001 | At 8-weeks: |
| At 8-weeks when compared to | A vs B, A vs C, A vs D, A vs E, A vs F, B vs C, B vs D, B |
| baseline: | vs E, B vs F, C vs D, C vs E, C vs F, D vs E, D vs F, E vs F- |
| Group A-ns | NS |
| Group B-*-p ≤ 0.0001 | At 12-weeks: |
| Group C-@-p ≤ 0.001 | A vs B, A vs C, A vs D-NS |
| Group D, Group E, Group F-*-p ≤ 0.0001 | A vs E-Ω-p ≤ 0.01 |
| At 12-weeks when compared to | A vs F-Ω-p ≤ 0.01 |
| baseline: | B vs C, B vs D, B vs E, B vs F, C vs D, C vs E, C vs F, D |
| Group A-ns | vs E, D vs F, E vs F-NS |
| Group B, Group C, Group D, Group | At 24-weeks: |
| E, Group F-*-p ≤ 0.0001 | A vs B-β-p ≤ 0.001 |
| At 24-weeks when compared to | A vs C-Ω-p ≤ 0.01 |
| baseline: | A vs D-‡-p ≤ 0.05 |
| Group A-ns | A vs E, A vs F-∞-p ≤ 0.0001 |
| Group B, Group C, Group D, Group | B vs C, B vs D, B vs E, B vs F, C vs D, C vs E, C vs F, D |
| E, Group F-*-p ≤ 0.0001 | vs E, D vs F, E vs F-NS |

### MENQOL Score at the End of 4, 8, 12 and 24-Weeks of Treatment and Between Group Analysis:

The data in the Table 2 depicts that group B, group D, group E, and group F have shown significant improvement in MENQOL score at 4, 8, 12, and 24-weeks when compared to baseline. Group C has not shown any significant effect at 4-weeks of treatment, but has shown effect at 8, 12, and 24-weeks when compared to baseline. Group A had not shown any significant effect at any of the weeks.

### Between Group Analysis at 4-Weeks:

With Placebo BD group: group A (Placebo BD) versus group B (Sensoril 125 mg BD)—No significance noted.

group A (Placebo BD) versus group C (Asparagus 125 mg BD)—No significance noted.

group A (Placebo BD) versus group D (Sen 125 mg+Asp 125 mg BD)—No significance noted.

group A (Placebo BD) versus group E (Sensoril 250 mg BD)—No significance noted.

group A (Placebo BD) versus group F (Asparagus 250 mg BD)—No significance noted.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—No significance noted.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It can be noted that all the groups have not shown any effect when compared to Placebo BD at 4-weeks. There was no significance between the treatment groups.

### Between Group Analysis at 8-Weeks:

### With Placebo BD Group:

group A (Placebo BD) versus group B (Sensoril 125 mg BD)—No significance noted.

group A (Placebo BD) versus group C (Asparagus 125 mg BD)—No significance noted

group A (Placebo BD) versus group D (Sen 125 mg+Asp 125 mg BD)—No significance noted.

group A (Placebo BD) versus group E (Sensoril 250 mg BD)—No significance noted.

group A (Placebo BD) versus group F (Asparagus 250 mg BD)—No significance noted.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—No significance noted.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It can be noted that all the groups have not shown any effect when compared to Placebo BD at 8-weeks. There was no significance between the treatment groups also.

### Between Group Analysis at 12-Weeks:

### With Placebo BD Group:

group A (Placebo BD) versus group B (Sensoril 125 mg BD)—No significance noted.

group A (Placebo BD) versus group C (Asparagus 125 mg BD)—No significance noted.

group A (Placebo BD) versus group D (Sen 125 mg+Asp 125 mg BD)—No significance noted.

group A (Placebo BD) versus group E (Sensoril 250 mg BD)

group E (Sensoril 250 mg BD) was better with a significance of p≤0.01.

group A (Placebo BD) versus group F (Asparagus 250 mg BD)

group F (Asparagus 250 mg BD) was better with a significance of p≤0.05.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—No significance noted.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD) No significance noted.

group E (Sensoril 250 mg BD) versus group F (Asparagus 250 mg BD)—No significance noted.

It can be noted that group E and group F have shown significant effect when compared to Placebo BD at 12-weeks. However, there was no significance between the treatment groups.

### Between Group Analysis at 24-Weeks:

### With Placebo BD Group:

group A (Placebo BD) versus group B (Sensoril 125 mg BD)

group B (Sensoril 125 mg BD) was better with a significance of p≤0.001.

group A (Placebo BD) versus group C (Asparagus 125 mg BD)

group C (Asparagus 125 mg BD) was better with a significance of p≤0.01.

group A (Placebo BD) versus group D (Sen 125 mg+Asp 125 mg BD)

group D (Sen 125 mg+Asp 125 mg BD) was better with a significance of p≤0.05.

group A (Placebo BD) versus group E (Sensoril 250 mg BD)

group E (Sensoril 250 mg BD) was better with a significance of p≤0.0001.

group A (Placebo BD) versus group F (Asparagus 250 mg BD)

group F (Asparagus 250 mg BD) was better with a significance of p≤0.0001.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—No significance noted.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It is seen that all the groups have shown a better effect than Placebo BD at 24-weeks. Group E and group F have shown significantly better effect when compared to Placebo BD at 24-weeks. However, there was no significance between the other treatment groups.

TABLE 3
Reflection Index (RI %) from baseline to 4, 8, 12 and 24-weeks of treatment (Mean ± SD)

| Treatment | | 4- | Abs | 8- | Abs | 12- | Abs | 24- | Abs |
|---|---|---|---|---|---|---|---|---|---|
| Groups | Baseline | weeks | Change | weeks | Change | weeks | Change | weeks | Change |
| Placebo BD | −3.29 ± | −3.28 ± | 0.02 ± | −3.29 ± | 0.00 ± | −3.32 ± | −0.03 ± | −3.32 ± | −0.03 ± |
| (A) | 0.70 | 0.80 ns | 0.39 | 0.87 ns | 0.56 | 0.82 ns | 0.57 | 0.74 ns | 0.62 |
| | | NS | | NS | | NS | | NS | |
| Sensoril | −3.41 ± | −3.50 ± | −0.10 ± | −3.55 ± | −0.15 ± | −5.34 ± | −1.93 ± | −6.35 ± | −2.94 ± |
| 125 mg BD | 1.05 | 1.12 ns | 0.26 | 1.01 ns | 0.26 | 1.17 * | 0.43 | 1.17 | 0.40 |
| (B) | | NS | | NS | | ∞ Ω | | * ∞ | |
| Asparagus | −3.21 ± | −3.25 ± | −0.04 ± | −3.39 ± | −0.19 ± | −4.39 ± | −1.18 ± | −4.98 ± | −1.78 ± |
| 125 mg BD | 0.94 | 0.99 ns | 0.25 | 0.86 ns | 0.34 | 0.83 * | 0.75 | 1.02 * | 0.87 |
| (C) | | NS | | NS | | Ω NS | | ∞ NS | |
| Sensoril | −3.29 ± | −3.37 ± | −0.08 ± | −3.60 ± | −0.30 ± | −4.11 ± | −0.82 ± | −4.44 ± | −1.15 ± |
| 125 mg BD + | 0.74 | 0.74 ns | 0.25 | 0.67 ns | 0.47 | 0.44* ‡ | 0.55 | 0.43 * | 0.59 |
| Asparagus | | NS | | NS | | ∞ | | Ω | |
| 125 mg BD | | | | | | | | | |
| (D) | | | | | | | | | |
| Sensoril | −3.37 ± | −3.50 ± | −0.13 ± | −3.70 ± | −0.33 ± | −7.08 ± | −3.71 ± | −8.66 ± | −5.30 ± |
| 250 mg BD | 0.89 | 0.92 ns | 0.27 | 0.76 * | 0.26 | 1.03 * | 0.73 | 0.95 * | 0.84 |
| (E) | | NS | | NS | | ∞ | | ∞ Ω | |
| Asparagus | −3.24 ± | −3.31 ± | −0.07 ± | −3.53 ± | −0.29 ± | −6.20 ± | −2.96 ± | −7.70 ± | −4.46 ± |
| 250 mg BD | 0.58 | 0.69 ns | 0.32 | 0.66 @ | 0.28 | 0.68 * | 0.54 | 0.79 * | 0.60 |
| (F) | | NS | | NS | | ∞ ‡ Ω | | ∞ | |

| | |
|---|---|
| At 4-weeks when compared to | BETWEEN GROUP ANALYSIS |
| baseline: | At 4-weeks: |
| Group A, Group B, Group C, Group | A vs B, A vs C, A vs D, A vs E, A vs F, B vs C, B vs D, B |
| D, Group E, Group F-ns | vs E, B vs F, C vs D, C vs E, C vs F, D vs E, D vs F, E vs F- |
| At 8-weeks when compared to | NS |
| baseline: | At 8-weeks: |
| Group A, Group B, Group C, Group | A vs B, A vs C, A vs D, A vs E, A vs F, B vs C, B vs D, B |
| D-ns | vs E, B vs F, C vs D, C vs E, C vs F, D vs E, D vs F, E vs F- |
| Group E-*-p ≤ 0.0001 | NS |
| Group F-@-p ≤ 0.001 | At 12-weeks: |
| At 12-weeks when compared to | A vs B-∞-p ≤ 0.0001 |
| baseline: | A vs C-Ω-p ≤ 0.01 |
| Group A-ns | A vs D-‡-p ≤ 0.05 |
| Group B, Group C, Group D, Group | A vs E, A vs F-∞-p ≤ 0.0001 |
| E, Group F-*-p ≤ 0.0001 | B vs C-Ω-p ≤ 0.01 |
| At 24-weeks when compared to | B vs D, B vs E-∞-p ≤ 0.0001 |
| baseline: | B vs F-‡-p ≤ 0.05 |
| Group A-ns | C vs D-NS |
| Group B, Group C, Group D, Group | C vs E, C vs F, D vs E, D vs F-∞-p ≤ 0.0001 |
| E, Group F-*-p < 0.0001 | E vs F-Ω-p ≤ 0.01 |
| | At 24-weeks: |
| | A vs B, A vs C-∞-p ≤ 0.0001 |
| | A vs D-Ω-p ≤ 0.01 |
| | A vs E, A vs F-∞-p ≤ 0.0001 |
| | B vs C, B vs D, B vs E, B vs F-∞-p ≤ 0.0001 |
| | C vs D-NS |
| | C vs E, C vs F, D vs E, D vs F-∞-p ≤ 0.0001 |
| | E vs F-Ω-p ≤ 0.01 |

### Reflection Index (RI %) at the End of 4, 8, 12 and 24-Weeks of Treatment and Between Group Analysis:

The data in the Table 3 depicts that all the treatment groups had not shown any effect at 4-weeks. At 8-weeks group A, group B, group C, and group D had not shown any effect. Whereas group E and group F have shown improvement in RI % at 8-weeks when compared to baseline. Group B, group C, group D, group E, and group F have shown significant improvement in RI % at 12 and 24-weeks when compared to baseline. Group A has not shown any significant effect at any of the weeks.

### Between Group Analysis at 4-Weeks:

### With Placebo BD Group:

group A (Placebo BD) versus group B (Sensoril 125 mg BD)—No significance noted.

group A (Placebo BD) versus group C (Asparagus 125 mg BD)—No significance noted.

group A (Placebo BD) versus group D (Sen 125 mg+Asp 125 mg BD)—No significance noted.

group A (Placebo BD) versus group E (Sensoril 250 mg BD)—No significance noted

group A (Placebo BD) versus group F (Asparagus 250 mg BD)—No significance noted.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—No significance noted.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It can be noted that all the groups have not shown any effect when compared to Placebo BD at 4-weeks. There was no significance between the treatment groups.

### Between Group Analysis at 8-Weeks:

### With Placebo BD Group:

group A (Placebo BD) versus group B (Sensoril 125 mg BD)—No significance noted.

group A (Placebo BD) versus group C (Asparagus 125 mg BD)—No significance noted.

group A (Placebo BD) versus group D (Sen 125 mg+Asp 125 mg BD)—No significance noted.

group A (Placebo BD) versus group E (Sensoril 250 mg BD)—No significance noted.

group A (Placebo BD) versus group F (Asparagus 250 mg BD)—No significance noted.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—No significance noted.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It can be noted that all the groups have not shown any effect when compared to Placebo BD at 8-weeks. There was no significance between the treatment groups also.

### Between Group Analysis at 12-Weeks:

### With Placebo BD Group:

group A (Placebo BD) versus group B (Sensoril 125 mg BD)

group B (Sensoril 125 mg BD) was better with a significance of p≤0.0001.

group A (Placebo BD) versus group C (Asparagus 125 mg BD)

group C (Asparagus 125 mg BD) was better with a significance of p≤0.01.

group A (Placebo BD) versus group D (Sen 125 mg+Asp 125 mg BD)

group D (Sen 125 mg+Asp 125 mg BD) was better with a significance of p≤0.05.

group A (Placebo BD) versus group E (Sensoril 250 mg BD)

group E (Sensoril 250 mg BD) was better with a significance of p≤0.0001.

group A (Placebo BD) versus group F (Asparagus 250 mg BD)

group F (Asparagus 250 mg BD) was better with a significance of p≤0.0001.

### With Active Treatment Groups:

group B (Sensoril 125 mg BD) versus group C (Asparagus 125 mg BD)

group B (Sensoril 125 mg BD) was better with a significance of p≤0.01.

group B (Sensoril 125 mg BD) versus group D (Sen 125 mg+Asp 125 mg BD)—group B (Sensoril 125 mg BD) was better with a significance of p≤0.0001.

group B (Sensoril 125 mg BD) versus group E (Sensoril 250 mg BD)—group E (Sensoril 250 BD) was better with a significance of p≤0.0001.

group B (Sensoril 125 mg BD) versus group F (Asparagus 250 mg BD)—group F (Asparagus 250 mg BD) was better with a significance of p≤0.05.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 BD) was better with a significance of p≤0.0001.

group C (Asparagus 125 mg BD) versus group F (Asparagus 250 mg BD)—group F (Asparagus 250 mg BD) was better with a significance of p≤0.0001.

group D (Sen 125 mg+Asp 125 mg BD) versus group E (Sensoril 250 mg BD)—group E (Sensoril 250 BD) was better with a significance of p≤0.0001.

group D (Sen 125 mg+Asp 125 mg BD) versus group F (Asparagus 250 mg BD)—group F (Asparagus 250 mg BD) was better with a significance of p≤0.0001.

group E (Sensoril 250 mg BD) versus group F (Asparagus 250 mg BD)—group E (Sensoril 250 BD) was better with a significance of p≤0.01.

It can be noted that all the treatment groups showed significant effect when compared to Placebo BD at 12-weeks. It can be noted that group B, group E and group F have shown significant effect when compared to other treatment groups at 12-weeks. However, group E has shown the best effect than all the other treatment groups.

### Between Group Analysis at 24-Weeks:

### With Placebo BD Group:

group A (Placebo BD) versus group B (Sensoril 125 mg BD)—group B (Sensoril 125 mg BD) was better with a significance of p≤0.0001.

group A (Placebo BD) versus group C (Asparagus 125 mg BD)—group C (Asparagus 125 mg BD) was better with a significance of p≤0.0001.

group A (Placebo BD) versus group D (Sen 125 mg+Asp 125 mg BD)—group D (Sen 125 mg+Asp 125 mg BD) was better with a significance of p≤0.01.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.0001.

### With Active Treatment Groups

group B (Sensoril 125 mg BD) versus group C (Asparagus 125 mg BD)—group B (Sensoril 125 mg BD) was better with a significance of p≤0.0001.

group B (Sensoril 125 mg BD) versus group D (Sen 125 mg+Asp 125 mg BD)—group B (Sensoril 125 mg BD) was better with a significance of p≤0.0001.

group B (Sensoril 125 mg BD) versus group E (Sensoril 250 mg BD)—group E (Sensoril 250 BD) was better with a significance of p≤0.0001.

group B (Sensoril 125 mg BD) versus group F (Asparagus 250 mg BD)—group F (Asparagus 250 mg BD) was better with a significance of p≤0.0001.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 BD) was better with a significance of p≤0.0001.

group C (Asparagus 125 mg BD) versus group F (Asparagus 250 mg BD)—group F (Asparagus 250 mg BD) was better with a significance of p≤0.0001.

group D (Sen 125 mg+Asp 125 mg BD) versus group E (Sensoril 250 mg BD)—group E (Sensoril 250 BD) was better with a significance of p≤0.0001.

group D (Sen 125 mg+Asp 125 mg BD) versus group F (Asparagus 250 mg BD)—group F (Asparagus 250 mg BD) was better with a significance of p≤0.0001.

group E (Sensoril 250 mg BD) versus group F (Asparagus 250 mg BD)—group E (Sensoril 250 BD) was better with a significance of p≤0.01.

It can be noted that all the treatment groups showed significant effect when compared to Placebo BD at 24-weeks. It is seen that group B, group E, and group F have shown significant effect when compared to other treatment groups at 24-weeks. However, group E has shown better effect than all the other treatment groups.

TABLE 4
Effect on Osteoprotegerin (OPG in pg/ml) OPG (Ref. Range: 93.75 to 6000
pg/ml) from baseline to 12 and 24-weeks of treatment (Mean ± SD)

| Treatment | | | | | |
|---|---|---|---|---|---|
| Groups | Baseline | 12-weeks | % change | 24-weeks | % change |
| Placebo BD | 1443.39 ± | 1444.31 ± | 0.14 ± | 1448.31 ± | 0.40 ± |
| (A) | 257.70 | 251.16 ns NS | 1.27 | 254.22 ns NS | 1.27 |
| Sensoril 125 mg | 1571.30 ± | 1858.67 ± | 20.41 ± | 2193.27 ± | 45.30 ± |
| BD | 425.80 | 392.23 $ ‡ | 11.14 | 342.11 * ∞ ‡ | 27.70 |
| (B) | | | | | |
| Asparagus 125 mg | 1670.58 ± | 1796.95 ± | 8.60 ± | 1890.25 ± | 14.27 ± |
| BD | 326.99 | 287.09 $ ‡ NS | 6.75 | 302.78 * β | 7.54 |
| (C) | | | | | |
| Sensoril 125 mg | 1605.34 ± | 1911.90 ± | 20.48 ± | 2072.35 ± | 31.43 ± |
| BD + Asparagus | 350.46 | 337.08 $ Ω NS | 8.72 | 316.73 * ∞ NS | 13.44 |
| 125 mg BD | | | | | |
| (D) | | | | | |
| Sensoril 250 mg | 1612.91 ± | 2082.73 ± | 30.67 ± | 2501.71 ± | 59.66 ± |
| BD | 397.35 | 429.04 $ ∞ NS | 11.77 | 350.04 * ∞ ‡ B | 23.14 |
| (E) | | | | | |
| Asparagus 250 mg | 1641.25 ± | 2016.31 ± | 24.54 ± | 2304.28 ± | 43.85 ± |
| BD | 404.18 | 421.31 $ ∞ NS | 11.99 | 420.22 * ∞ Ω NS ‡ | 20.70 |
| (F) | | | | | |

* p ≤ 0.0001,
@ p ≤ 0.001,
$ p ≤ 0.01,
# p ≤ 0.05,
ns nonsignificant
∞ p ≤ 0.0001,
β p ≤ 0.001,
Ω p ≤ 0.01,
‡ p ≤ 0.05,
NS nonsignificant

| | |
|---|---|
| At 12-weeks when compared to baseline: | BETWEEN GROUP ANALYSIS |
| Group A - ns | At 12-weeks: |
| Group B, Group C, Group D, Group E, | A vs B, A vs C - ‡ - p ≤ 0.05 |
| Group F - $ - p ≤ 0.01 | A vs D - Ω - p ≤ 0.01 |
| At 24-weeks when compared to baseline: | A vs E, A vs F - ∞ - p ≤ 0.0001 |
| Group A-ns | B vs C, B vs D, B vs E, B vs F - NS |
| Group B, Group C, Group D, Group E, | C vs D, C vs E, C vs F - NS |
| Group F - * - p ≤ 0.0001 | D vs E, D vs F - NS |
| | E vs F - NS |
| | At 24-weeks: |
| | A vs B - ∞ - p ≤ 0.0001 |
| | A vs C - β - p ≤ 0.001 |
| | A vs D, A vs E, A vs F - ∞-p ≤ 0.0001 |
| | B vs C - ‡ - p ≤ 0.05 |
| | B vs D - NS |
| | B vs E - ‡ - p ≤ 0.05 |
| | B vs F - NS |
| | C vs D - NS |
| | C vs E - ∞ - p ≤ 0.0001 |
| | C vs F - Ω - p ≤ 0.01 |
| | D vs E - β - p ≤ 0.001 |
| | D vs F - NS |
| | E vs F - ‡ - p ≤ 0.05 |

Osteoprotegerin (OPG in pg/ml) at the End of 12 and 24 Weeks of Treatment and Between Group Analysis:

The data in the Table 4 depicts that group B, group C, group D, group E, and group F have shown significant improvement in OPG values at 12-weeks and 24-weeks when compared to baseline. Group A has not shown any effect.

### Between Group Analysis at 12-Weeks:

### With Placebo BD Group:

group A (Placebo BD) versus group B (Sensoril 125 mg BD)—group B (Sensoril 125 mg BD) was better with a significance of p≤0.05.

group A (Placebo BD) versus group C (Asparagus 125 mg BD)—group C (Asparagus 125 mg BD) was better with a significance of p≤0.05.

group A (Placebo BD) versus group D (Sen 125 mg+Asp 125 mg BD)—group D (Sen 125 mg+Asp 125 mg BD) was better with a significance of p≤0.01.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.0001.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—No significance noted.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It can be noted that all the groups have shown significant effect when compared to Placebo BD at 12-weeks. However, there was no significance between the treatment groups.

### Between Group Analysis at 24-Weeks:

### With Placebo BD Group:

group A (Placebo BD) versus group B (Sensoril 125 mg BD)—group B (Sensoril 125 mg BD) was better with a significance of p≤0.0001.

group A (Placebo BD) versus group C (Asparagus 125 mg BD)—group C (Asparagus 125 mg BD) was better with a significance of p≤0.001.

group A (Placebo BD) versus group D (Sen 125 mg+Asp 125 mg BD)—group D (Sen 125 mg+Asp 125 mg BD) was better with a significance of p≤0.0001.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.0001.

### With Active Treatment Groups:

group B (Sensoril 125 mg BD) versus group C (Asparagus 125 mg BD)—group B (Sensoril 125 mg BD) was better with a significance of p≤0.05.

group B (Sensoril 125 mg BD) versus group D (Sen 125 mg+Asp 125 mg BD)—No significance noted.

group B (Sensoril 125 mg BD) versus group E (Sensoril 250 mg BD)—group E (Sensoril 250 BD) was better with a significance of p≤0.05.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 BD) was better with a significance of p≤0.0001.

group C (Asparagus 125 mg BD) versus group F (Asparagus 250 mg BD)—group F (Asparagus 250 mg BD) was better with a significance of p≤0.01.

group D (Sen 125 mg+Asp 125 mg BD) versus group E (Sensoril 250 mg BD)—group E (Sensoril 250 BD) was better with a significance of p≤0.001.

group D (Sen 125 mg+Asp 125 mg BD) versus group F (Asparagus 250 mg BD)—No significance noted.

group E (Sensoril 250 mg BD) versus group F (Asparagus 250 mg BD)—group E (Sensoril 250 BD) was better with a significance of p 0.05.

It is seen that all the groups have shown a better effect than Placebo BD at 24-weeks. Group B and group F have shown better effect than group C. Whereas group E has shown significantly better effect than all the treatment groups.

TABLE 5
Effect on Soluble receptor activator of nuclear factor- kappaB Ligand
(sRANKL) (Ref. Range: 78.125 to 5000 pg/ml) from baseline to
12 and 24-weeks of treatment (Mean ± SD)

| Treatment | | | | | |
|---|---|---|---|---|---|
| Groups | Baseline | 12-weeks | % change | 24-weeks | % Change |
| Placebo | 1631.40 ± 287.30 | 1629.85 ± 286.18 | −0.11 ± 0.24 | 1630.700 ± 287.55 | −0.05 ± 0.12 |
| BD (A) | | ns NS | | ns NS | |
| Sensoril 125 mg | 1685.55 ± 261.15 | 1454.75 ± 230.50 | −13.66 ± 3.48 | 1034.85 ± 156.81 | −38.28 ± 5.78 |
| BD (B) | | * NS | | * ∞ | |
| Asparagus 125 mg | 1755.54 ± 325.59 | 1545.05 ± 316.69 | −12.27 ± 3.87 | 1312.85 ± 258.76 | −24.67 ± 9.70 |
| BD (C) | | * NS | | * ‡ | |
| Sensoril 125 mg | 1440.62 ± 427.27 | 1290.38 ± 406.08 | −10.75 ± 4.26 | 1192.24 ± 404.11 | −18.12 ± 7.23 |
| BD + Asparagus | | * Ω NS | | * β NS | |
| 125 mg BD (D) | | | | | |
| Sensoril 250 mg | 1714.77 ± 291.72 | 1222.55 ± 284.37 | −29.17 ± 8.18 | 758.32 ± 234.00 | −56.44 ± 9.74 |
| BD (E) | | * β NS | | * ∞ ‡ | |
| Asparagus 250 mg | 1736.35 ± 306.91 | 1403.70 ± 343.28 | −19.87 ± 9.80 | 1149.30 ± 373.58 | −34.42 ± 16.76 |
| BD (F) | | * ‡ NS | | * ∞ NS β | |

* - p ≤ 0.0001, @ - p ≤ 0.001, $ - p ≤ 0.01, # - p ≤ 0.05, ns - nonsignificent
∞ - p ≤ 0.0001, β - p ≤ 0.001, Ω - p ≤ 0.01, ‡ - p ≤ 0.05, NS - nonsignificant

| | |
|---|---|
| At 12-weeks when compared to baseline: | BETWEEN GROUP ANALYSIS |
| Group A - ns | At 12-weeks: |
| Group B, Group C, Group D, Group E, | A vs B, A vs C - NS |
| Group F - * - p ≤ 0.0001 | A vs D - Ω - p ≤ 0.01 |
| At 24-weeks when compared to baseline: | A vs E - β - p ≤ 0.001 |
| Group A - ns | A vs F - ‡ - p ≤ 0.05 |
| Group B, Group C, Group D, Group E, | B vs C, B vs D, B vs E, B vs F - NS |
| Group F - * - p ≤ 0.0001 | C vs D - NS |
| | C vs E - ‡ - p ≤ 0.05 |
| | C vs F - NS |
| | D vs E, D vs F - NS |
| | E vs F - NS |
| | At 24-weeks: |
| | A vs B - ∞ - p ≤ 0.0001 |
| | A vs C - ‡ - p ≤ 0.05 |
| | A vs D - β - p ≤ 0.001 |
| | A vs E, A vs F - ∞ - p ≤ 0.0001 |
| | B vs C - ‡ - p ≤ 0.05 |
| | B vs D - NS |
| | B vs E - ‡ - p ≤ 0.05 |
| | B vs F - NS |
| | C vs D - NS |
| | C vs E - ∞ - p ≤ 0.0001 |
| | C vs F - NS |
| | D vs E - ∞ - p ≤ 0.0001 |
| | D vs F - NS |
| | E vs F - β - p ≤ 0.001 |

Soluble Receptor Activator of Nuclear Factor-kappaB Ligand (sRANKL in pmol/L) at the End of 12 and 24 Weeks of Treatment and Between Group Analysis:

The data in the Table 5 depicts that group B, group C, group D, group E, and group F have shown significant improvement in RANKL values at 12-weeks and 24-weeks treatment when compared to baseline. Group A has not shown any effect.

### Between Group Analysis at 12-Weeks:

### With Placebo BD Group:

group A (Placebo BD) versus group B (Sensoril 125 mg BD)—No significance noted.

group A (Placebo BD) versus group C (Asparagus 125 mg BD)—No significance noted.

group A (Placebo BD) versus group D (Sen 125 mg+Asp 125 mg BD)—group D (Sen 125 mg+Asp 125 mg BD) was better with a significance of p≤0.01.

group A (Placebo BD) versus group E (Sensoril 250 mg BD)—group E (Sensoril 250 mg BD) was better with a significance of p≤0.001.

group A (Placebo BD) versus group F (Asparagus 250 mg BD)—group F (Asparagus 250 mg BD) was better with a significance of p≤0.05.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 mg BD) was better with a significance of p≤0.05.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It can be noted that all the groups have shown significant effect when compared to Placebo BD at 12-weeks. Group E was better than group C. However, there was no significance between the other treatment groups.

### Between Group Analysis at 24-Weeks:

### With Placebo BD Group:

group A (Placebo BD) versus group B (Sensoril 125 mg BD)—group B (Sensoril 125 mg BD) was better with a significance of p≤0.0001.

group A (Placebo BD) versus group C (Asparagus 125 mg BD)—group C (Asparagus 125 mg BD) was better with a significance of p≤0.05.

group A (Placebo BD) versus group D (Sen 125 mg+Asp 125 mg BD)—group D (Sen 125 mg+Asp 125 mg BD) was better with a significance of p≤0.001.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.0001.

### With Active Treatment Groups:

group B (Sensoril 125 mg BD) versus group C (Asparagus 125 mg BD)—group B (Sensoril 125 mg BD) was better with a significance of p≤0.05.

group B (Sensoril 125 mg BD) versus group D (Sen 125 mg+Asp 125 mg BD)—No significance noted.

group B (Sensoril 125 mg BD) versus group E (Sensoril 250 mg BD)—group E (Sensoril 250 BD) was better with a significance of p≤0.05.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 BD) was better with a significance of p≤0.0001.

group C (Asparagus 125 mg BD) versus group F (Asparagus 250 mg BD)—No significance noted.

group D (Sen 125 mg+Asp 125 mg BD) versus group E (Sensoril 250 mg BD)—group E (Sensoril 250 BD) was better with a significance of p≤0.0001.

group D (Sen 125 mg+Asp 125 mg BD) versus group F (Asparagus 250 mg BD)—No significance noted.

group E (Sensoril 250 mg BD) versus group F (Asparagus 250 mg BD)—group E (Sensoril 250 BD) was better with a significance of p≤0.001.

It is seen that all the groups have shown a better effect than Placebo BD at 24-weeks. Group B has shown better effect than group C. Whereas group E has shown significantly better effect than all the treatment groups. There was no significance between the other treatment groups.

TABLE 6
Effect on C-terminal telopeptide-1 (CTX-1) (Ref. Range: 0.149 to 2.276
ng/ml) from baseline to 12 and 24-weeks of treatment (Mean ± SD)

| Treatment | | | | | |
|---|---|---|---|---|---|
| Groups | Baseline | 12-weeks | % change | 24-weeks | % Change |
| Placebo | 1.836 ± 0.407 | 1.834 ± 0.404 | −0.07 ± 1.26 | 1.827 ± 0.406 | −0.53 ± 1.20 |
| BD (A) | | ns NS | | ns NS | |
| Sensoril 125 mg | 1.902 ± 0.36 | 1.517 ± 0.38 | −19.92 ± 14.08 | 1.106 ± 0.25 | −40.62 ± 13.84 |
| BD (B) | | * NS | | * ∞ Ω | |
| Asparagus 125 mg | 1.756 ± 0.30 | 1.651 ± 0.28 | −5.86 ± 4.67 | 1.49 ± 0.26 | −14.50 ± 8.73 |
| BD (C) | | @ NS | | * ‡ | |
| Sensoril 125 mg | 1.863 ± 0.37 | 1.656 ± 0.33 | −10.84 ± 7.90 | 1.31 ± 0.26 | −28.14 ± 15.26 |
| BD + Asparagus | | * NS | | * β NS | |
| 125 mg BD (D) | | | | | |
| Sensoril 250 mg | 1.950 ± 0.42 | 1.457 ± 0.29 | −23.78 ± 12.33 | 0.766 ± 0.31 | −60.51 ± 13.82 |
| BD (E) | | * Ω NS | | * ∞ ‡ | |
| Asparagus 250 mg | 1.926 ± 0.47 | 1.595 ± 0.45 | −16.36 ± 16.45 | 1.304 ± 0.55 | −32.60 ± 22.73 |
| BD (F) | | * NS | | * β NS ∞ | |

* - p ≤ 0.0001, @ - p ≤ 0.001, $ - p ≤ 0.01, # - p ≤ 0.05, ns - nonsignificent
∞ - p ≤ 0.0001, β - p ≤ 0.001, Ω - p ≤ 0.01, ‡ - p ≤ 0.05, NS - nonsignificant

| | |
|---|---|
| At 12-weeks when compared to baseline: | BETWEEN GROUP ANALYSIS |
| Group A - ns | At 12-weeks: |
| Group B - * - p ≤ 0.0001 | A vs B, A vs C, A vs D - NS |
| Group C - @ - p ≤ 0.001 | A vs E - Q - p ≤ 0.01 |
| Group D, Group E - *- p ≤ 0.0001 | A vs F - NS |
| Group F - @ - p ≤ 0.001 | B vs C, B vs D, B vs E, B vs F - NS |
| At 24-weeks when compared to baseline: | C vs D, C vs E, C vs F - NS |
| Group A - ns | D vs E, D vs F - NS |
| Group B, Group C, Group D, Group E, | E vs F - NS |
| Group F - * - p < 0.0001 | At 24-weeks: |
| | A vs B - ∞ - p ≤ 0.0001 |
| | A vs C - ‡ - p ≤ 0.05 |
| | A vs D - β - p ≤ 0.001 |
| | A vs E - ∞ - p ≤ 0.0001 |
| | A vs F - β - p ≤ 0.001 |
| | B vs C - Ω - p ≤ 0.01 |
| | B vs D - NS |
| | B vs E - ‡ - p ≤ 0.05 |
| | B vs F - NS |
| | C vs D - NS |
| | Cvs E - ∞ - p ≤ 0.0001 |
| | C vs F - NS |
| | D vs E - ∞ - p ≤ 0.0001 |
| | D vs F - NS |
| | E vs F - ∞ - p < 0.0001 |

C-Terminal Telopeptide-1 (CTX-1 in ng/mL) at the End of 12 and 24 Weeks of Treatment and Between Group Analysis:

The data in the Table 6 depicts that group B, group C, group D, group E, and group F have shown significant improvement in CTX-1 values at 12-weeks and 24-weeks when compared to baseline. Group A has not shown any effect.

### Between Group Analysis at 12-Weeks:

### With Placebo BD Group:

group A (Placebo BD) versus group B (Sensoril 125 mg BD)—No significance noted.

group A (Placebo BD) versus group C (Asparagus 125 mg BD)—No significance noted.

group A (Placebo BD) versus group D (Sen 125 mg+Asp 125 mg BD)—No significance noted.

group A (Placebo BD) versus group E (Sensoril 250 mg BD)—group E (Sensoril 250 mg BD) was better with a significance of p≤0.01.

group A (Placebo BD) versus group F (Asparagus 250 mg BD)—No significance noted.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—No significance noted.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It can be noted that all the groups have shown significant effect when compared to Placebo BD at 12-weeks. However, there was no significance between the other treatment groups.

### Between Group Analysis at 24-Weeks:

### With Placebo BD Group:

group A (Placebo BD) versus group B (Sensoril 125 mg BD)—group B (Sensoril 125 mg BD) was better with a significance of p≤0.0001.

group A (Placebo BD) versus group C (Asparagus 125 mg BD)—group C (Asparagus 125 mg BD) was better with a significance of p≤0.05.

group A (Placebo BD) versus group D (Sen 125 mg+Asp 125 mg BD)—group D (Sen 125 mg+Asp 125 mg BD) was better with a significance of p≤0.001.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.001.

### With Active Treatment Groups:

group B (Sensoril 125 mg BD) versus group C (Asparagus 125 mg BD)—group B (Sensoril 125 mg BD) was better with a significance of p≤0.01.

group B (Sensoril 125 mg BD) versus group D (Sen 125 mg+Asp 125 mg BD)—No significance noted.

group B (Sensoril 125 mg BD) versus group E (Sensoril 250 mg BD)—group E (Sensoril 250 BD) was better with a significance of p≤0.05.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 BD) was better with a significance of p≤0.0001.

group C (Asparagus 125 mg BD) versus group F (Asparagus 250 mg BD)—No significance noted.

group D (Sen 125 mg+Asp 125 mg BD) versus group E (Sensoril 250 mg BD)—group E (Sensoril 250 BD) was better with a significance of p≤0.0001.

group D (Sen 125 mg+Asp 125 mg BD) versus group F (Asparagus 250 mg BD)—No significance noted.

group E (Sensoril 250 mg BD) versus group F (Asparagus 250 mg BD)—group E (Sensoril 250 BD) was better with a significance of p≤0.001.

It is seen that all the groups have shown a better effect than Placebo BD at 24-weeks. Group B has shown better effect than group C. Whereas group E has shown significantly better effect than all the treatment groups. There was no significance between the other treatment groups.

TABLE 7
Effect on Bone Alkaline Phosphatase (BAP) (Ref. Range: 7 to 90 ng/mL)
from baseline to 12 and 24-weeks of treatment (Mean ± SD)

| Treatment | | | | | |
|---|---|---|---|---|---|
| Groups | Baseline | 12-weeks | % change | 24-weeks | % Change |
| Placebo | 23.86 ± 6.82 | 23.82 ± 6.82 | −0.14 ± 1.38 | 23.75 ± 6.75 | −0.37 ± 1.31 |
| BD (A) | | ns NS | | ns NS | |
| Sensoril 125 mg | 22.07 ± 4.88 | 18.01 ± 4.07 | −18.03 ± 7.15 | 13.57 ± 3.55 | −37.60 ± 12.78 |
| BD (B) | | * $ | | * ∞ ‡ | |
| Asparagus 125 mg | 20.78 ± 5.33 | 19.12 ± 5.42 | −8.34 ± 7.14 | 18.43 ± 5.48 | −12.05 ± 7.29 |
| BD (C) | | @ NS | | * ‡ | |
| Sensoril 125 mg | 19.78 ± 3.59 | 16.95 ± 3.42 | −14.21 ± 7.00 | 14.41 ± 3.26 | −26.91 ± 10.71 |
| BD + Asparagus | | * β NS | | * ∞ NS | |
| 125 mg BD (D) | | | | | |
| Sensoril 250 mg | 20.99 ± 4.66 | 15.84 ± 4.05 | −24.83 ± 6.87 | 9.11 ± 1.22 | −55.26 ± 7.93 |
| BD (E) | | * ∞ NS | | * ∞ ‡ Ω β | |
| Asparagus 250 mg | 24.89 ± 7.74 | 21.42 ± 7.63 | −14.53 ± 10.04 | 17.96 ± 6.71 | −28.22 ± 12.61 |
| BD (F) | | * NS | | * Ω NS | |

* - p ≤ 0.0001, @ - p ≤ 0.001, $ - p ≤ 0.01, # - p ≤ 0.05, ns - nonsignificent
∞ - p ≤ 0.0001, β - p ≤ 0.001, Ω - p ≤ 0.01, ‡ - p ≤ 0.05, NS - nonsignificant

| | |
|---|---|
| At 12-weeks when compared to baseline: | BETWEEN GROUP ANALYSIS |
| Group A - ns | At 12-weeks: |
| Group B - * - p ≤ 0.0001 | A vs B - Ω - p ≤ 0.01 |
| Group C - @ - p ≤ 0.001 | A vs C - NS |
| Group D, Group E, Group F - | A vs D - β - p ≤ 0.001 |
| * - p ≤ 0.0001 | A vs E - ∞ - p ≤ 0.0001 |
| At 24-weeks when compared to baseline: | A vs F - NS |
| Group A - n - s | B vs C, B vs D, B vs E, B vs F - NS |
| Group B, Group C, Group D, Group E, | C vs D, C vs E, C vs F - NS |
| Group F - * - p ≤ 0.0001 | D vs E, D vs F - NS |
| | E vs F - NS |
| | At 24-weeks: |
| | A vs B - ∞ - p ≤ 0.0001 |
| | A vs C - ‡ - p ≤ 0.05 |
| | A vs D, A vs E - ∞ - p ≤ 0.0001 |
| | A vs F - Ω - p ≤ 0.01 |
| | B vs C - ‡ - p ≤ 0.05 |
| | B vs D - NS |
| | B vs E - ‡ - p ≤ 0.05 |
| | B vs F - NS |
| | C vs D - NS |
| | C vs E - ∞ - p ≤ 0.0001 |
| | C vs F - NS |
| | D vs E - Ω - p ≤ 0.01 |
| | D vs F - NS |
| | E vs F - β - p ≤ 0.001 |

Bone Alkaline Phosphatase (BAP in μg/L) at the End of 12 and 24 Weeks of Treatment and Between Group Analysis:

The data in the Table 7 depicts that group B, group C, group D, group E, and group F have shown significant improvement in BAP values at 12-weeks and 24-weeks when compared to baseline. Group A has not shown any effect.

### Between Group Analysis at 12-Weeks:

### With Placebo BD Group:

group A (Placebo BD) versus group B (Sensoril 125 mg BD)—group B (Sensoril 125 mg BD) was better with a significance of p≤0.01.

group A (Placebo BD) versus group C (Asparagus 125 mg BD)—No significance noted.

group A (Placebo BD) versus group D (Sen 125 mg+Asp 125 mg BD)—group D (Sen 125 mg+Asp 125 mg BD) was better with a significance of p≤0.001.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—No significance noted.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—No significance noted.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It can be noted that group B, group D and group E have shown significant effect when compared to Placebo BD at 12-weeks. However, there was no significance between the other treatment groups.

### Between Group Analysis at 24-Weeks:

### With Placebo BD Group:

group A (Placebo BD) versus group B (Sensoril 125 mg BD)—group B (Sensoril 125 mg BD) was better with a significance of p≤0.0001.

group A (Placebo BD) versus group C (Asparagus 125 mg BD)—group C (Asparagus 125 mg BD) was better with a significance of p≤0.05.

group A (Placebo BD) versus group D (Sen 125 mg+Asp 125 mg BD)—group D (Sen 125 mg+Asp 125 mg BD) was better with a significance of p≤0.0001.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.01.

### With Active Treatment Groups:

group B (Sensoril 125 mg BD) versus group C (Asparagus 125 mg BD)—group B (Sensoril 125 mg BD) was better with a significance of p≤0.05.

group B (Sensoril 125 mg BD) versus group D (Sen 125 mg+Asp 125 mg BD)—No significance noted.

group B (Sensoril 125 mg BD) versus group E (Sensoril 250 mg BD)—group E (Sensoril 250 BD) was better with a significance of p≤0.05.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 BD) was better with a significance of p≤0.0001.

group C (Asparagus 125 mg BD) versus group F (Asparagus 250 mg BD)—No significance noted.

group D (Sen 125 mg+Asp 125 mg BD) versus group E (Sensoril 250 mg BD)—group E (Sensoril 250 BD) was better with a significance of p≤0.01.

group D (Sen 125 mg+Asp 125 mg BD) versus group F (Asparagus 250 mg BD)—No significance noted.

group E (Sensoril 250 mg BD) versus group F (Asparagus 250 mg BD)—group E (Sensoril 250 BD) was better with a significance of p≤0.001.

It is seen that all the groups have shown a better effect than Placebo BD at 24-weeks. Group B has shown better effect than group C. Whereas group E has shown significantly better effect than all the treatment groups. There was no significance between the other treatment groups.

TABLE 8
Effect on Nitric oxide (NO in μM/L) from baseline
to 12 and 24-weeks of treatment (Mean ± SD)

| Treatment | | | | | Absolute |
|---|---|---|---|---|---|
| Groups | Baseline | 12-weeks | % change | 24-weeks | Change |
| Placebo | 31.95 ± 3.00 | 31.96 ± 3.07 | 0.02 ± 1.45 | 32.02 ± 3.04 | 0.23 ± 1.95 |
| BD (A) | | ns NS | | ns NS | |
| Sensoril 125 mg | 34.45 ± 3.71 | 37.12 ± 4.11 | 7.75 ± 1.78 | 39.32 ± 4.39 | 14.11 ± 2.99 |
| BD (B) | | $ Ω NS | | * β NS | |
| Asparagus 125 mg | 34.92 ± 3.92 | 37.39 ± 4.09 | 7.20 ± 3.87 | 39.00 ± 3.94 | 11.92 ± 4.55 |
| BD (C) | | $ ‡ NS | | * Ω NS | |
| Sensoril 125 mg | 33.68 ± 3.48 | 36.63 ± 3.58 | 8.96 ± 1.59 | 38.75 ± 3.65 | 15.21 ± 3.33 |
| BD + Asparagus | | $ ‡ NS | | * Ω NS | |
| 125 mg BD (D) | | | | | |
| Sensoril 250 mg | 36.40 ± 3.841 | 41.73 ± 2.63 | 15.40 ± 8.78 | 45.05 ± 2.34 | 25.07 ± 14.40 |
| BD (E) | | $ ∞ Ω ‡ @ | | * ∞ Ω β | |
| Asparagus 250 mg | 32.57 ± 2.84 | 36.21 ± 2.76 | 11.36 ± 4.62 | 38.90 ± 3.01 | 19.61 ± 4.98 |
| BD (F) | | $ ‡ | | * Ω | |

* - p ≤ 0.0001, @ - p ≤ 0.001, $ - p ≤ 0.01, # - p ≤ 0.05, ns - nonsignificent
∞ - p ≤ 0.0001, β - p ≤ 0.001, Ω - p ≤ 0.01, ‡ - p ≤ 0.05, NS - nonsignificant

| | |
|---|---|
| At 12-weeks when compared to baseline: | BETWEEN GROUP ANALYSIS |
| Group A - ns | At 12-weeks: |
| Group B, Group C, Group D, Group E, | A vs B, A vs C - Ω - p ≤ 0.01 |
| Group F - $ - p ≤ 0.01 | A vs D - ‡ - p ≤ 0.05 |
| At 24-weeks when compared to baseline: | A vs E - ∞ - p ≤ 0.0001 |
| Group A - ns | A vs F - ‡ - p ≤ 0.05 |
| Group B, Group C, Group D, Group E, | B vs C, B vs D - NS |
| Group F - * - p ≤ 0.0001 | B vs E - Ω - p ≤ 0.01 |
| | B vs F - NS |
| | C vs D - NS |
| | C vs E - β - p ≤ 0.001 |
| | C vs F - NS |
| | D vs E - Ω - p ≤ 0.01 |
| | D vs F - NS |
| | E vs F - β - p ≤ 0.001 |
| | At 24-weeks: |
| | A vs B - β - p ≤ 0.001 |
| | A vs C, A vs D - Ω - p ≤ 0.01 |
| | A vs E - ∞ - p ≤ 0.0001 |
| | A vs F - Ω - p ≤ 0.01 |
| | B vs C, B vs D - NS |
| | B vs E - Ω - p ≤ 0.01 |
| | B vs F - NS |
| | C vs D - NS |
| | C vs E - β - p ≤ 0.001 |
| | C vs F - NS |
| | D vs E - β - p ≤ 0.001 |
| | D vs F - NS |
| | E vs F - β - p ≤ 0.001 |

### Nitric Oxide (NO) at the End of 12 and 24 Weeks of Treatment and Between Group Analysis:

The data in the Table 8 depicts that group B, group C, group D, group E, and group F have shown significant improvement in NO values at 12-weeks and 24-weeks when compared to baseline. Group A has not shown any significant effect.

### Between Group Analysis at 12-Weeks:

### With Placebo BD Group:

group A (Placebo BD) versus group B (Sensoril 125 mg BD)—group B (Sensoril 125 mg BD) was better with a significance of p≤0.01.

group A (Placebo BD) versus group C (Asparagus 125 mg BD)—group C (Asparagus 125 mg BD) was better with a significance of p≤0.01.

group A (Placebo BD) versus group D (Sen 125 mg+Asp 125 mg BD)—group D (Sen 125 mg+Asp 125 mg BD) was better with a significance of p≤0.05.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.05.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—group E (Sensoril 250 mg BD) was better with a significance of p≤0.01.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 mg BD) was better with a significance of p≤0.001.

group C (Asparagus 125 mg BD) versus group F (Asparagus 250 mg BD)—No significance noted.

group D (Sen 125 mg+Asp 125 mg BD) versus group E (Sensoril 250 mg BD)—group E (Sensoril 250 mg BD) was better with a significance of p≤0.01.

group D (Sen 125 mg+Asp 125 mg BD) versus group F (Asparagus 250 mg BD)—No significance noted.

group E (Sensoril 250 mg BD) versus group F (Asparagus 250 mg BD)—group E (Sensoril 250 mg BD) was better with a significance of p≤0.001.

It can be noted that group B, group C, group D and group E have shown significant effect when compared to Placebo BD at 12-weeks. There was no significance between the other treatment groups, however, group E has shown better response than other groups.

### Between Group Analysis at 24-Weeks:

### With Placebo BD Group:

group A (Placebo BD) versus group B (Sensoril 125 mg BD)—group B (Sensoril 125 mg BD) was better with a significance of p≤0.001.

group A (Placebo BD) versus group C (Asparagus 125 mg BD)—group C (Asparagus 125 mg BD) was better with a significance of p≤0.01.

group A (Placebo BD) versus group D (Sen 125 mg+Asp 125 mg BD)—group D (Sen 125 mg+Asp 125 mg BD) was better with a significance of p≤0.01.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.01.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—group E (Sensoril 250 BD) was better with a significance of p≤0.01.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 BD) was better with a significance of p≤0.001.

group C (Asparagus 125 mg BD) versus group F (Asparagus 250 mg BD)—No significance noted.

group D (Sen 125 mg+Asp 125 mg BD) versus group E (Sensoril 250 mg BD)—group E (Sensoril 250 BD) was better with a significance of p≤0.001.

group D (Sen 125 mg+Asp 125 mg BD) versus group F (Asparagus 250 mg BD)—No significance noted.

group E (Sensoril 250 mg BD) versus group F (Asparagus 250 mg BD)—group E (Sensoril 250 BD) was better with a significance of p≤0.001.

It is seen that all the groups have shown a better effect than Placebo BD at 24-weeks. Group B has shown better effect than group C. Whereas group E has shown significantly better effect than all the treatment groups.

TABLE 9
Effect on Glutathione (GSH in μmol/L) from
baseline to 12 and 24-weeks of treatment (Mean ± SD)

| Treatment Groups | Baseline | 12-weeks | % change | 24-weeks | % change |
|---|---|---|---|---|---|
| Placebo | 575.60 ± 9.49 | 575.64 ± 9.59 | 0.01 ± 0.10 | 575.61 ± 9.54 | 0.02 ± 0.05 |
| BD (A) | | ns NS | | ns NS | |
| Sensoril 125 mg | 576.28 ± 20.10 | 606.99 ± 19.46 | 5.36 ± 1.92 | 619.25 ± 18.08 | 7.49 ± 1.88 |
| BD (B) | | $ ∞ NS | | *∞ NS | |
| Asparagus 125 mg | 574.01 ± 15.15 | 596.46 ± 16.80 | 3.91 ± 1.04 | 610.16 ± 16.05 | 6.31 ± 1.26 |
| BD (C) | | $ Ω NS | | * β NS | |
| Sensoril 125 mg | 575.61 ± 9.88 | 597.95 ± 8.96 | 3.89 ± 1.24 | 612.92 ± 9.60 | 6.49 ± 1.28 |
| BD + Asparagus | | $ β NS | | * Ω NS | |
| 125 mg BD (D) | | | | | |
| Sensoril 250 mg | 578.61 ± 22.95 | 631.24 ± 9.84 | 9.22 ± 3.60 | 645.39 ± 14.49 | 11.73 ± 3.74 |
| BD (E) | | $ ∞ Ω | | * ∞ Ω | |
| Asparagus 250 mg | 566.84 ± 12.43 | 601.51 ± 12.11 | 6.13 ± 1.26 | 610.35 ± 7.58 | 7.71 ± 1.68 |
| BD (F) | | $ Ω | | * Ω | |

* - p ≤ 0.0001, @ - p ≤ 0.001, $ - p ≤ 0.01, # - p ≤ 0.05, ns - nonsignificent
∞ - p ≤ 0.0001, β - p ≤ 0.001, Ω - p ≤ 0.01, ‡ - p ≤ 0.05, NS - nonsignificant

| | |
|---|---|
| At 12-weeks when compared to baseline: | BETWEEN GROUP ANALYSIS |
| Group A - ns | At 12-weeks: |
| Group B, Group C, Group D, Group E, | A vs B - ∞ - p ≤ 0.0001 |
| Group F - $ - p ≤ 0.01 | A vs C - Ω - p ≤ 0.01 |
| At 24-weeks when compared to baseline: | A vs D - β - p ≤ 0.001 |
| Group A - ns | A vs E - ∞ - p ≤ 0.0001 |
| Group B, Group C, Group D, Group E, | A vs F - Ω - p ≤ 0.01 |
| Group F - * - p ≤ 0.0001 | B vs C, B vs D - NS |
| | B vs E - Ω - p ≤ 0.01 |
| | B vs F - NS |
| | C vs D - NS |
| | C vs E - ∞ - p ≤ 0.0001 |
| | C vs F - NS |
| | D vs E - ∞ - p ≤ 0.0001 |
| | D vs F - NS |
| | E vs F - ∞ - p ≤ 0.0001 |
| | At 24-weeks: |
| | A vs B - ∞ - p ≤ 0.0001 |
| | A vs C - β - p ≤ 0.001 |
| | A vs D - Ω - p ≤ 0.01 |
| | A vs E - ∞ - p ≤ 0.0001 |
| | A vs F - Ω - p ≤ 0.01 |
| | B vs C, B vs D - NS |
| | B vs E - Ω - p ≤ 0.01 |
| | B vs F - NS |
| | C vs D - NS |
| | C vs E - ∞ - p ≤ 0.0001 |
| | C vs F - NS |
| | D vs E - ∞ - p ≤ 0.0001 |
| | D vs F - NS |
| | E vs F - ∞ - p < 0.0001 |

### Glutathione (GSH) at the End of 12 and 24 Weeks of Treatment and Between Group Analysis:

The data in the Table 9 depicts that group B, group C, group D, group E, and group F have shown significant improvement in GSH values at 12-weeks and 24-weeks when compared to baseline. Group A has not shown any effect.

### Between Group Analysis at 12-Weeks:

### With Placebo BD Group:

group A (Placebo BD) versus group B (Sensoril 125 mg BD)—group B (Sensoril 125 mg BD) was better with a significance of p≤0.0001.

group A (Placebo BD) versus group C (Asparagus 125 mg BD)—group C (Asparagus 125 mg BD) was better with a significance of p≤0.01.

group A (Placebo BD) versus group D (Sen 125 mg+Asp 125 mg BD)—group D (Sen 125 mg+Asp 125 mg BD) was better with a significance of p≤0.001.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.01.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—group E (Sensoril 250 mg BD) was better with a significance of p≤0.01.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 mg BD) was better with a significance of p≤0.0001.

group C (Asparagus 125 mg BD) versus group F (Asparagus 250 mg BD)—No significance noted.

group D (Sen 125 mg+Asp 125 mg BD) versus group E (Sensoril 250 mg BD)—group E (Sensoril 250 mg BD) was better with a significance of p≤0.0001.

group D (Sen 125 mg+Asp 125 mg BD) versus group F (Asparagus 250 mg BD)—No significance noted.

group E (Sensoril 250 mg BD) versus group F (Asparagus 250 mg BD)—group E (Sensoril 250 mg BD) was better with a significance of p≤0.0001.

It can be noted that all the treatment groups have shown significant effect when compared to Placebo BD at 12-weeks. Group E was better when compared to other groups. There was no significance between the other treatment groups at 12-weeks.

### Between Group Analysis at 24-Weeks:

### With Placebo BD Group:

group A (Placebo BD) versus group B (Sensoril 125 mg BD)—group B (Sensoril 125 mg BD) was better with a significance of p≤0.0001.

group A (Placebo BD) versus group C (Asparagus 125 mg BD)—group C (Asparagus 125 mg BD) was better with a significance of p≤0.001.

group A (Placebo BD) versus group D (Sen 125 mg+Asp 125 mg BD)—group D (Sen 125 mg+Asp 125 mg BD) was better with a significance of p≤0.01.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.01.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—group E (Sensoril 250 BD) was better with a significance of p≤0.01.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 BD) was better with a significance of p≤0.0001.

group C (Asparagus 125 mg BD) versus group F (Asparagus 250 mg BD)—No significance noted.

group D (Sen 125 mg+Asp 125 mg BD) versus group E (Sensoril 250 mg BD)—group E (Sensoril 250 BD) was better with a significance of p≤0.0001.

group D (Sen 125 mg+Asp 125 mg BD) versus group F (Asparagus 250 mg BD)—No significance noted.

group E (Sensoril 250 mg BD) versus group F (Asparagus 250 mg BD)—group E (Sensoril 250 BD) was better with a significance of p≤0.0001.

It is seen that all the groups have shown a better effect than Placebo BD at 24-weeks. Group E has shown significantly better effect than all the treatment groups. There was no significance between the other treatment groups at 24-weeks.

TABLE 10
Effect on Malondialdehyde (MDA in μM/L from
baseline to 12 and 24-weeks of treatment (Mean ± SD)

| Treatment Groups | Baseline | 12-weeks | % change | 24-weeks | % Change |
|---|---|---|---|---|---|
| Placebo | 3.74 ± 0.38 | 3.75 ± 0.36 | 0.26 ± 1.88 | 3.74 ± 0.35 | 0.02 ± 2.04 |
| BD (A) | | ns NS | | ns NS | |
| Sensoril 125 mg | 3.56 ± 0.48 | 3.35 ± 0.43 | −5.70 ± 4.60 | 3.17 ± 0.45 | −10.65 ± 7.80 |
| BD (B) | | $ NS | | * NS | |
| Asparagus 125 mg | 3.45 ± 0.49 | 3.39 ± 0.47 | −1.66 ± 2.35 | 3.33 ± 0.46 | −3.39 ± 2.71 |
| BD (C) | | ns NS | | * NS | |
| Sensoril 125 mg | 3.38 ± 0.49 | 3.35 ± 0.49 | −0.68 ± 1.07 | 3.22 ± 0.47 | −4.75 ± 1.67 |
| BD + Asparagus | | $ Ω NS | | * Ω NS | |
| 125 mg BD (D) | | | | | |
| Sensoril 250 mg | 3.30 ± 0.43 | 2.96 ± 0.27 | −9.78 ± 5.44 | 2.74 ± 0.09 | −15.82 ± 8.61 |
| BD (E) | | @ ∞ Ω NS | | * ∞ Ω β | |
| Asparagus 250 mg | 3.27 ± 0.41 | 3.19 ± 0.38 | −2.59 ± 1.25 | 3.13 ± 0.36 | −4.26 ± 2.54 |
| BD (F) | | $ Ω | | * Ω | |

* - p ≤ 0.0001, @ - p ≤ 0.001, $ - p ≤ 0.01, # - p ≤ 0.05, ns - nonsignificent
∞ - p ≤ 0.0001, β - p ≤ 0.001, Ω - p ≤ 0.01, ‡ - p ≤ 0.05, NS - nonsignificant

| | |
|---|---|
| At 12-weeks when compared to baseline: | BETWEEN GROUP ANALYSIS |
| Group A - ns | At 12-weeks: |
| Group B - $ - p ≤ 0.01 | A vs B, A vs C - NS |
| Group C - ns | A vs D - Ω - p ≤ 0.01 |
| Group D - $ - p ≤ 0.01 | A vs E - ∞ - p ≤ 0.0001 |
| Group E - @ - p ≤ 0.001 | A vs F - Ω - p ≤ 0.01 |
| Group F - $ - p ≤ 0.01 | B vs C, B vs D, B vs E, B vs F - NS |
| At 24-weeks when compared to baseline: | C vs D - NS |
| Group A - ns | C vs E - Q - p ≤ 0.01 |
| Group B, Group C, Group D, Group E, | C vs F - NS |
| Group F - * - p ≤ 0.0001 | D vs E, D vs F - NS |
| | E vs F - NS |
| | At 24-weeks: |
| | A vs B - Ω - p ≤ 0.01 |
| | A vs C - NS |
| | A vs D - Ω - p ≤ 0.01 |
| | A vs E - ∞ - p ≤ 0.0001 |
| | A vs F - Ω - p ≤ 0.01 |
| | B vs C, B vs D - NS |
| | B vs E - Ω - p ≤ 0.01 |
| | B vs F - NS |
| | C vs D - NS |
| | C vs E - β - p ≤ 0.001 |
| | C vs F - NS |
| | D vs E - Ω - p ≤ 0.01 |
| | D vs F - NS |
| | E vs F - Ω - p ≤ 0.01 |

### Malondialdehyde (MDA) at the End of 12 and 24 Weeks of Treatment and Between Group Analysis:

The data in the Table 10 depicts that group B, group D, group E, and group F have shown significant improvement in MDA values at 12-weeks when compared to baseline, whereas group A and group C have not shown any effect. At 24-weeks all the treatment groups have shown significant effect when compared to baseline. Group A has not shown any effect.

### Between Group Analysis at 12-Weeks:

### With Placebo BD Group:

group A (Placebo BD) versus group B (Sensoril 125 mg BD)—No significance noted.

group A (Placebo BD) versus group C (Asparagus 125 mg BD)—No significance noted.

group A (Placebo BD) versus group D (Sen 125 mg+Asp 125 mg BD)—group D (Sen 125 mg+Asp 125 mg BD) was better with a significance of p≤0.01.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.01.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—No significance noted.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 mg BD) was better with a significance of p≤0.01.

group C (Asparagus 125 mg BD) versus group F (Asparagus 250 mg BD)—No significance noted.

group D (Sen 125 mg+Asp 125 mg BD) versus group E (Sensoril 250 mg BD)—No significance noted.

group D (Sen 125 mg+Asp 125 mg BD) versus group F (Asparagus 250 mg BD)—No significance noted.

group E (Sensoril 250 mg BD) versus group F (Asparagus 250 mg BD)—No significance noted.

It can be noted that all the treatment groups have shown better response when compared to Placebo BD group at 12-weeks. There was no significance between the other treatment groups, however group E was better than group C.

### Between Group Analysis at 24-Weeks:

### With Placebo BD Group:

group A (Placebo BD) versus group B (Sensoril 125 mg BD)—group B (Sensoril 125 mg BD) was better with a significance of p≤0.01.

group A (Placebo BD) versus group C (Asparagus 125 mg BD)—No significance noted.

group A (Placebo BD) versus group D (Sen 125 mg+Asp 125 mg BD)—group D (Sen 125 mg+Asp 125 mg BD) was better with a significance of p≤0.01.

group A (Placebo BD) versus group E (Sensoril 250 mg BD)—group E (Sensoril 250 mg BD) was better with a significance of p≤0.0001.

group A (Placebo BD) versus group F (Asparagus 250 mg BD)—group F (Asparagus 250 mg BD) was better with a significance of p≤0.01.

### With Active Treatment Groups:

group B (Sensoril 125 mg BD) versus group C (Asparagus 125 mg BD)—No significance noted.

group B (Sensoril 125 mg BD) versus group D (Sen 125 mg+Asp 125 mg BD)—No significance noted.

group B (Sensoril 125 mg BD) versus group E (Sensoril 250 mg BD)—group E (Sensoril 250 BD) was better with a significance of p≤0.01.

group B (Sensoril 125 mg BD) versus group F (Asparagus 250 mg BD)—No significance noted.

group C (Asparagus 125 mg BD) versus group D (Sen 125 mg+Asp 125 mg BD)—No significance noted.

group C (Asparagus 125 mg BD) versus group E (Sensoril 250 mg BD)—group E (Sensoril 250 BD) was better with a significance of p≤0.001.

group C (Asparagus 125 mg BD) versus group F (Asparagus 250 mg BD)—No significance noted.

group D (Sen 125 mg+Asp 125 mg BD) versus group E (Sensoril 250 mg BD)—group E (Sensoril 250 BD) was better with a significance of p≤0.01.

group D (Sen 125 mg+Asp 125 mg BD) versus group F (Asparagus 250 mg BD)—No significance noted.

group E (Sensoril 250 mg BD) versus group F (Asparagus 250 mg BD)—group E (Sensoril 250 BD) was better with a significance of p≤0.001.

It is seen that all the groups have shown a better effect than Placebo BD at 24-weeks. Group E has shown significantly better effect than all the treatment groups. There was no significance between the other treatment groups.

TABLE 11
Effect on platelet aggregation using ADP (10 μM/ml)
from baseline to end of 12 weeks of treatment (Mean ± SD):

| Groups | Baseline | 24 weeks | % inhibition |
|---|---|---|---|
| Placebo BD (A) | 64.80 ± 8.89 | 67.35 ± 9.02 | 0.44 ± 1.11 |
| Sensoril 125 mg BD (B) | 65.85 ± 9.26 | 66.50 ± 10.49 | 2.11 ± 2.50 |
| Asparagus 125 mg BD (C) | 66.80 ± 11.49 | 66.90 ± 12.79 | 2.98 ± 3.54 |
| Sensoril 125 mg BD + | 69.29 ± 9.94 | 68.71 ± 9.07 | 3.28 ± 2.94 |
| Asparagus 125 mg BD (D) | | | |
| Sensoril 250 mg BD (E) | 68.77 ± 8.78 | 67.14 ± 5.89 | 3.81 ± 3.04 |
| Asparagus 250 mg BD (F) | 69.50 ± 7.03 | 66.85 ± 6.33 | 4.43 ± 3.42 |

TABLE 12
Effect on platelet aggregation using collagen (2 μg/ml)
from baseline to end of 12 weeks of treatment (Mean ± SD):

| Groups | Baseline | 24 weeks | % inhibition |
|---|---|---|---|
| Placebo BD (A) | 71.25 ± 11.02 | 74.25 ± 10.62 | 0.68 ± 1.28 |
| Sensoril 125 mg BD (B) | 69.85 ± 10.38 | 71.05 ± 9.80 | 1.47 ± 1.79 |
| Asparagus 125 mg BD (C) | 68.55 ± 10.08 | 68.95 ± 9.79 | 1.64 ± 1.88 |
| Sensoril 125 mg BD + | 70.86 ± 9.77 | 69.95 ± 8.38 | 2.23 ± 2.06 |
| Asparagus 125 mg BD (D) | | | |
| Sensoril 250 mg BD (E) | 71.32 ± 8.91 | 71.00 ± 8.59 | 2.87 ± 2.26 |
| Asparagus 250 mg BD (F) | 73.45 ± 9.62 | 73.20 ± 8.92 | 1.89 ± 1.83 |

### Platelet Aggregation Percent Inhibition (% Inhibition) at the End of 24 Weeks of Treatment:

The normal range of platelet aggregation using ADP (10 μM/ml) and collagen (2 μg/ml) is between 60% and 90%. To show if the drug affects platelet aggregation, there should be more than 30% change in percent inhibition. As depicted in Tables 11 and 12 the percent inhibition is minimal, indicating that all the treatment groups have no effect on platelet aggregation.

### Safety Assessments:

All safety haematological and biochemical parameters performed as per visit schedule were within normal limits with all the six treatment groups at baseline and the end of the treatment. One subject in group E (Sensoril 250 mg BD) and three in group F (Asparagus 250 mg BD) reported mild GI disturbance, which subsided with symptomatic treatment. None of the subject in either group discontinued the study due to adverse events.

### CONCLUSION

In the present study, we observed that extracts of Withania somnifera, Asparagus racemosus, and their combination significantly improved biomarkers of bone health i.e., CTX-1, BAP, OPG and sRANKL when compared to baseline and placebo. Significant improvement in the biomarkers of stress MDA, NO, and GSH along with endothelial function as estimated by improvement in reflection index (RI) was also observed. Significant improvement in quality of life as measured by MENQOL questionnaire was also observed when compared to baseline and placebo.

Improvement in quality of life as measured by MENQOL score improved significantly from week 4 in all the groups except group A (Placebo BD) and group C (Asparagus 125 mg BD) though a trend was seen in these two groups. MENQOL scores improved in all the groups from week 8 with maximum effect at 24-weeks. Again, though a trend was observed with group A (Placebo BD) it was not statistically significant. Between-group analysis, it was observed all the groups were better when compared to placebo at 12 and 24-weeks. No significance was noted between other treatment groups.

At 4-weeks no statistically significant improvement in endothelial function as estimated by improvement in reflection index (RI) was seen with any of the treatment groups, though a trend was seen. Effect on endothelial function as estimated by improvement in reflection index (RI) was seen from week 8 in group E (Sensoril 250 mg BD) and group F (Asparagus 250 mg BD). All the treatment groups had shown significant improvement in RI at 12 and 24-weeks. Though a trend was observed with group A (Placebo BD) it was not statistically significant. Between-group analysis, it was observed all the groups were better when compared to placebo at 12 and 24-weeks. However, group B (Sensoril 125 mg BD), group E (Sensoril 250 mg BD), and group F (Asparagus 250 mg BD) were better than other treatment groups at 12 and 24-weeks. Group E (Sensoril 250 mg BD) had shown the best response of all.

Significant improvement in the biomarkers of bone health i.e., OPG, sRANKL, CTX-1, BAP, and OPG with group B (Sensoril 125 mg BD), group C (Asparagus 125 mg BD), group D (Sen 125 mg BD+Asp 125 mg BD), group E (Sensoril 250 mg BD), and group F (Asparagus 250 mg BD) at 12 and 24-weeks when compared to baseline was observed. The maximum benefit was seen at 24-weeks. No significant effect though a trend was seen with group A (Placebo BD). Between-group analysis, it was observed all the groups were better when compared to placebo at 12-weeks. No significance was seen between treatment groups at 12-weeks. However, group B (Sensoril 125 mg BD), group E (Sensoril 250 mg BD), and group F (Asparagus 250 mg BD) were better than other treatment groups at 24-weeks. Group E (Sensoril 250 mg BD) had shown the best response of all.

The biomarkers of stress—NO, GSH, and MDA significantly improved with all the treatment groups when compared to baseline at 12 and 24-weeks. Group A (Placebo BD) had not shown any significant improvement in the stress biomarkers though a trend was observed. On between-group analysis, it was observed all the groups were better when compared to placebo at 12 and 24-weeks in improving NO and GSH levels. Whereas improvement in MDA was seen at 24-weeks. Group E (Sensoril 250 mg BD) had shown the best response in improving the stress biomarkers at both 12 and 24-weeks.

No effect on platelet aggregation was observed with any of the treatment groups. The medications used in the study were well tolerated. No serious adverse events were reported. None of the subjects discontinued the study due to any adverse events, which suggests the favorable safety profile of the treatments used in the study.

The findings of this study suggest that daily intake of extracts of Withania somnifera and Asparagus racemosus may have beneficial effect on bone health, oxidative stress, and quality of life health in postmenopausal women. Further studies are warranted to substantiate the beneficial effect of these herbal products in postmenopausal women.

### REFERENCES

1. Franco O H, Chowdhury R, Troup J, Voortman T, Kunutsor S, Kavousi M et al. Use of Plant-Based Therapies and Menopausal Symptoms: A Systematic Review and Meta-analysis. JAMA. 2016; 315(23):2554-63.
2. Pandey A K, Gupta A, Tiwari M, Prasad S, Pandey A N, Yadav P K et al. Impact of stress on female reproductive health disorders: Possible beneficial effects of shatavari (Asparagus racemosus). Biomed Pharmacother. 2018; 103:46-49.
3. Modi M. B., Donga S. B., Dei L. Clinical evaluation of Ashokarishta, AshwagandhaChurna and PravalPishti in the management of menopausal syndrome. Ayu. 2012; 33(4):511-516.
4. RaminNasimiDoostAzgomi, Afshar Zomorrodi, Hossein Nazemyieh, et al., “Effects of Withania somnifera on Reproductive System: A Systematic Review of the Available Evidence,” BioMed Research International, 2018; 17.
5. Ashajyothi V, Rao, Pippalla S, Satyavati D. Asparagus racemosus—A Phytoestrogen. IJPT. 2009; 1(1): 36-47.
6. Priyanka Kulkarni, Pramod Khobragade. Phytoestrogens Medicinal Herbs—Safe and Effective Alternative to Hormone Replacement Therapy in Menopausal Syndrome. J. res. tradit. med. 2016; 2(5): 147-150.
7. Pingali Usharani et al. Evaluation of a Highly Standardized Withania Somnifera Extract on Endothelial Dysfunction and Biomarkers of Oxidative Stress in Patients with Type 2 Diabetes Mellitus: A Randomized, Double Blind, Placebo Controlled Study. Int. J. Ayur. Pharma Research, 2014; 2(3): 22-32.
8. Gannon J M, Brar J, Rai A, Chengappa K N R. Effects of a standardized extract of Withania somnifera (Ashwagandha) on depression and anxiety s ymptoms in persons with schizophrenia participating ina randomized, placebo-controlled clinical trial. Ann Clin Psychiatry. 2019 May; 31(2):123-129.
9. Chengappa K N R, Brar J S, Gannon J M, Schlicht P J. Adjunctive Use of a Standardized Extract of Withania somnifera (Ashwagandha) to Treat Symptom Exacerbation in Schizophrenia: A Randomized, Double-Blind, Placebo-Controlled Study. J Clin Psychiatry. 2018 Jul. 10; 79(5).
10. Chengappa K N, Bowie C R, Schlicht P J, Fleet D, Brar J S, Jindal R. Randomized placebo-controlled adjunctive study of an extract of Withania somnifera for cognitive dysfunction in bipolar disorder. J Clin Psychiatry. 2013 November; 74(11):1076-83.
11. Chowienezyk P J, Kelly R P, MacCallum H, Millasseau S C, Tomas L G A, Gosling R G. Photoplethysmographic assessment of pulse wave reflection: blunted response to endothelium dependent beta2-adrenergic vasodilation in type II diabetes mellitus. J of American College of Cardiology. 1999; 34(7): 2007-2014.
12. Naidu M U R, Sridhar Y, Usha Rani P, Mateen A A. Comparison of two β2 adrenoceptor agonists by different routes of administration to assess human endothelial function. Indian J Pharmacol. 2007; 39 (3):168-169.
13. Millaesseau, S. C., Kelly, R. P., Ritter, J. M., Chowienczyk, P. J. Determination of age-related increases in large artery stiffness by digital pulse contour analysis. Clinical Science. 2002; 103:371-377.
14. Vidyasagar, J., Karunaka, N., Reddy, M S., Rajnarayan, K., Surender, T., Krishna D R. Oxidative stress and antioxidant status in acute organophosphorus insecticide poisoning. Indian J of Pharmacol. 2004; 36(2): 76-79.
15. Katrina, M M., Michael, G E., David, A. W. A rapid, simple spectrophotometrically method for simultaneous detection of nitrate and nitrite. Biology and Chemistry. 2001; 5(1): 62-71.
16. Elman, G. L. Tissue sulfhydryl groups. Arch BiochemBiophys. 1959; 82:70-77.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of treating postmenopausal women comprising the steps of:
a. providing a composition comprising an Asparagus racemosus extract;
b. administering an effective amount of the composition to the subject to deliver the Asparagus racemosus to the subject's bloodstream and bodily tissues; and
c. wherein the Asparagus racemosus acts in said bloodstream and/or bodily tissues to significantly improve biomarkers of bone health including C-terminal telopeptide-1 (CTX-1), Bone Alkaline Phosphatase (BAP), Osteoprotegerin (OPG) or serum receptor activator of nuclear factor-κB ligand (sRANKL).

2. The method of claim 1, further comprising significantly increases biomarkers of stress and endothelial function; wherein the biomarkers of stress are Malondialdehyde (MDA), Nitric Oxide (NO), or Glutathione (GSH).

3. The method of claim 1, further comprising significantly improving endothelial function as estimated by improvement in reflection index (RI).

4. The method of claim 1, wherein said Asparagus racemosus is administered daily to the subject.

5. The method of claim 1, wherein said composition is a dietary supplement.

6. The method of claim 5, wherein the dietary supplement includes about 125 mg or about 250 mg extract of Asparagus racemosus.

7. A method of treating postmenopausal women comprising the steps of:
a. providing a composition comprising Withania somnifera;
b. administering an effective amount of the composition to the subject to deliver the Withania somnifera to the subject's bloodstream and bodily tissues; and
c. wherein the Withania somnifera acts in said bloodstream and/or bodily tissues to significantly improve biomarkers of bone health including C-terminal telopeptide-1 (CTX-1), Bone Alkaline Phosphatase (BAP), Osteoprotegerin (OPG) and serum receptor activator of nuclear factor-κB ligand (sRANKL).

8. The method of claim 7, further comprising significantly increases biomarkers of stress and endothelial function; wherein the biomarkers of stress are Malondialdehyde (MDA), Nitric Oxide (NO), or Glutathione (GSH).

9. The method of claim 7, further comprising significantly improving endothelial function as estimated by improvement in reflection index (RI).

10. The method of claim 7, wherein said Withania somnifera is administered daily to the subject.

11. The method of claim 7, wherein said composition is a dietary supplement.

12. The method of claim 11, wherein the dietary supplement includes about 125 mg or about 250 mg extract of Withania somnifera.

13. A method of treating postmenopausal women comprising the steps of:
a. providing a composition comprising Withania somnifera and Asparagus racemosus;
b. administering an effective amount of the composition to the subject to deliver the Withania somnifera and Asparagus racemosus to the subject's bloodstream and bodily tissues; and
c. wherein the Withania somnifera and Asparagus racemosus acts in said bloodstream and/or bodily tissues to significantly improve biomarkers of bone health including C-terminal telopeptide-1 (CTX-1), Bone Alkaline Phosphatase (BAP), Osteoprotegerin (OPG) and serum receptor activator of nuclear factor-κB ligand (sRANKL).

14. The method of claim 13, further comprising significantly increases biomarkers of stress and endothelial function; wherein the biomarkers of stress are Malondialdehyde (MDA), Nitric Oxide (NO), or Glutathione (GSH).

15. The method of claim 13, further comprising significantly improving endothelial function as estimated by improvement in reflection index (RI).

16. The method of claim 13, wherein said Withania somnifera and Asparagus racemosus is administered daily to the subject.

17. The method of claim 13, wherein said composition is a dietary supplement.

18. The method of claim 17, wherein the dietary supplement includes about 125 mg or about 250 mg extract of Withania somnifera and Asparagus racemosus.
